(19) United States
(12) Patent Application Publication
(10) Pub. No.: US 2026/0166306 A1
(43) Pub. Date: Jun. 18, 2026

(54) IMPLANTABLE MEDICAL DEVICE FOR USE WITH OR HAVING RECORDING ELECTRODE

(71) Applicant: Biogen MA Inc., Cambridge, MA (US)

(72) Inventors: Daniel J. Abrams, Denver, CO (US); Matias Maturana, Melbourne (AU); Ross Thomas, Melbourne (AU); Christopher Smith, Melbourne (AU)

(73) Assignee: Biogen MA Inc., Cambridge, MA (US)

(21) Appl. No.: 19/529,504

(22) Filed: Feb. 4, 2026

Related U.S. Application Data
(63) Continuation of application No. 18/108,414, filed on Feb. 10, 2023, now Pat. No. 12,605,537.
(60) Provisional application No. 63/310,288, filed on Feb. 15, 2022.

(51) Int. Cl. A61N 1/05; A61B 5/00; A61B 5/293

(52) U.S. Cl. CPC A61N 1/0539; A61B 5/293; A61B 5/6864; A61B 5/6868

(57) ABSTRACT

A burr hole device is configured to receive a catheter and a cable of a sheath. The sheath is configured to receive a portion of the catheter and has an electrode. When the catheter and sheath are implanted with the burr hole device, the catheter and electrode of the sheath are implanted in a brain. Systems and apparatuses may include the burr hole device and/or a cranial port device, the catheter, and the sheath.

### CROSS REFERENCE TO RELATED APPLICATION

This application claims the benefit of priority to U.S. Provisional Patent Application No. 63/310,288, filed on 15 Feb. 2022, the disclosure of which is incorporated herein by reference in its entirety.

### FIELD

The present disclosure relates to, among other things, devices, assemblies, and systems for use with, or including, devices having a recording electrode for detecting brain activity data; particularly for use with an implantable catheter, as well as methods associated therewith.

### INTRODUCTION

Catheters and associated devices have been employed to deliver therapeutic agents to compartments of the brain comprising cerebral spinal fluid (CSF) or for withdrawing CSF from the brain. The catheters may be used to drain CSF from the brain, such as with a ventriculoperitoneal shunt or external ventricular drain; to percutaneously deliver therapeutic fluid to, or withdraw CSF from, the CSF space, such as with Ommaya or Rickman's reservoir; or to infuse therapeutic agent to the CSF space, such as with an implantable infusion device. Surgical procedures to implant the catheters and associated devices are invasive. For example, surgical placement of the catheter alone requires a burr hole to be drilled through the skull and the catheter or an introducer to be advanced through brain tissue to reach the CSP-containing compartment.

In some cases, such catheters and associated devices may be used to monitor a patient's health or therapy progression. For example, if the device includes an access port for withdrawing CSF through catheter, the withdrawn CSF may be evaluated, for example, to determine whether the patient has a bacterial, fungal, or viral infection or to determine whether a delivered therapeutic agent is present in the CSF at effective concentrations. However, monitoring of electrical brain signals, which may be important for monitoring the therapy, a condition being treated, or a brain state, is not possible with such devices.

Brain activity may be monitored in such patients by using a separate system to record brain activity from the scalp. Such electroencephalogram (EEG) recordings may be useful for periodic monitoring of the patient's brain activity but are not suitable for long-term, continuous monitoring for patients that are not confined to a health care facility. In addition, the signal provided by such EEG recordings tends to be noisy and of lower quality due to signal attenuation through the skull and scalp.

### SUMMARY

The present disclosure relates to, among other things, devices and systems configured for use with a catheter for delivering therapeutic agents to the brain or withdrawing, shunting, or draining CSF from the brain. The devices and systems may include a sheath configured to receive the catheter. The sheath may include an electrode configured to be intracranially positioned for recording brain activity data. The devices and systems may include a burr hole apparatus configured to receive the catheter and the sheath or portions thereof.

The devices and systems described herein may facilitate intracranial implantation of an electrode for recording brain activity data with a catheter for delivering fluid to or withdrawing, shunting, or draining fluid from a brain of a subject.

In an aspect, the present disclosure describes a burr hole device. The burr hole device is configured to receive a catheter for delivering fluid to or withdrawing, shunting, or draining fluid from a brain of a subject and to receive a cable of a sheath, where a conductor of the cable is electrically coupled to an electrode of the sheath.

In embodiments, the burr hole device includes a body having a top surface and a bottom surface and defining a lumen extending from the top surface to the bottom surface. The lumen is configured to receive a catheter configured to infuse fluid to, or withdraw fluid from, a brain of a subject. At least a portion of the bottom surface is configured to be disposed on a surface of a skull adjacent to a burr hole in the skull of the subject. The body further defines a top groove along the top surface. The top groove extends from the lumen towards a lateral edge of the top surface of the body. The top groove is configured to receive the catheter. The body further defines a bottom groove along the bottom surface. The bottom groove extends from a lateral edge of the bottom surface end towards the lumen. The bottom groove is configured to receive a cable. The cable may be a cable of a sheath device configured to receive the lead, wherein the sheath device has an electrode to which a conductor of the cable is electrically coupled.

In embodiments, the burr hole device includes a body having a top surface and a bottom surface and defining a lumen extending from the top surface to the bottom surface. The lumen is configured to receive a catheter configured to infuse fluid to, or withdraw fluid from, a brain of a subject. At least a portion of the bottom surface is configured to be disposed on a surface of a skull adjacent to a burr hole in the skull of the subject. The body further defines a first top groove along the top surface. The first top groove extends from the lumen towards a lateral edge of the top surface of the body. The first top groove is configured to receive the catheter. The body further defines a second top groove along the top surface. The second top groove extends from the lumen towards a lateral edge of the top surface of the body. The second top groove is configured to receive a cable. The cable may be a cable of a sheath device configured to receive the lead, wherein the sheath device has an electrode to which a conductor of the cable is electrically coupled.

In embodiments, the burr hole device includes a body having a top surface and a bottom surface and defining a lumen extending from the top surface to the bottom surface. The lumen is configured to receive a catheter configured to infuse fluid to, or withdraw fluid from, a brain of a subject. At least a portion of the bottom surface is configured to be disposed on a surface of a skull adjacent to a burr hole in the skull of the subject. The body further defines a top groove along the top surface. The top groove extends from the lumen towards a lateral edge of the top surface of the body. The top groove comprises a lower groove portion and an upper groove portion, the lower groove portion having a smaller diametric dimension than the upper groove portion. The lower groove portion is configured to receive a cable. The upper groove portion is configured to receive the catheter such that the catheter is positioned over the cable when the both the cable and the catheter are received in the top groove. The cable may be a cable of a sheath device configured to receive the lead, wherein the sheath device has an electrode to which a conductor of the cable is electrically coupled.

In embodiments, the burr hole device defines an upper flange portion and a lower portion. The lower portion is configured to be placed within the burr hole, and the upper flange portion is configured to be disposed on the surface of the skull. The lower portion of the body may be configured to have a clearance of 1 millimeter or less when disposed in the burr hole.

An assembly may include the burr hole device, the catheter, and a sheath. The sheath may include a sheath body defining a sleeve lumen configured to receive the catheter, (ii) a recording electrode disposed on the sheath body, (iii) the cable, and (iv) a conductor electrically coupled to the recording electrode and extending in the cable. The catheter may be a therapeutic fluid delivery catheter, a catheter of a ventriculoperitoneal (VP) shunt, a catheter of an external ventricular drain (EVD), a catheter for aspirating CSF, or the like. The assembly may comprise a VP shunt, an EVD, an infusion device, which may be an implantable infusion device, configured to connect to the therapeutic fluid delivery catheter, or the like.

A method may include disposing the burr hole device in or about a burr hole of a subject; inserting the catheter through the lumen of the burr hole device; inserting the catheter through the sheath lumen; implanting a distal end of the catheter in a brain of the subject; implanting the recording electrode of the sheath beneath the skull of the subject; inserting the cable of the sheath in the bottom groove, the second top groove, or the bottom portion of the top groove, depending on which burr hole device is employed; and inserting the catheter in the top groove, the first top groove, or the top portion of the top groove.

In an aspect, the present disclosure describes a burr hole device. The burr hole device is configured to be inserted into a burr hole of a subject and to be secured against a side of the burr hole.

In an embodiment, a burr hole device comprises a body having a top surface and a bottom surface and defining a lumen extending from the top surface to the bottom surface. The lumen may be configured to receive one or both of a cable of a sheath or lead and a catheter. The catheter may be configured to infuse fluid to, or withdraw fluid from, a brain of a subject. At least a portion of the body of the burr hole device is configured to be inserted into a burr hole. The burr hole device comprises an expansion member that may be deployed when the body is in the burr hole. The expansion member is configured to expand against a side of the burr hole to anchor the body within the burr hole. The expansion member is deployable from a retracted state to an expanded state. When the expansion member is in the retracted state, the body is slidably receivable in the burr hole. The expansion member may be operatively coupled to a user actuatable member that, when actuated, causes the expansion member to adapt the expanded state. The burr hole device may optionally comprise a portion configured to be disposed on a surface of the skull. For example, the configured to be disposed on a surface of the skull may be an upper flange portion. The portion configured to be disposed on a surface of the skull may comprise a groove for receiving the cable.

An assembly may include the burr hole device, the catheter, and a sheath. The sheath may include a sheath body defining a sleeve lumen configured to receive the catheter, (ii) a recording electrode disposed on the sheath body, (iii) the cable, and (iv) a conductor electrically coupled to the recording electrode and extending in the cable. The catheter may be a therapeutic fluid delivery catheter, a catheter of a ventriculoperitoneal (VP) shunt, a catheter of an external ventricular drain (EVD), a catheter for aspirating CSF, or the like. The assembly may comprise a VP shunt, an EVD, an infusion device, which may be an implantable infusion device, configured to connect to the therapeutic fluid delivery catheter, or the like.

A method may include disposing the burr hole device in or about a burr hole of a subject; inserting the catheter through the lumen of the burr hole device; inserting the catheter through the sheath lumen; implanting a distal end of the catheter in a brain of the subject; implanting the recording electrode of the sheath beneath the skull of the subject; inserting the cable of the sheath in the bottom groove, the second top groove, or the bottom portion of the top groove, depending on which burr hole device is employed; and inserting the catheter in the top groove, the first top groove, or the top portion of the top groove.

In an aspect, the present disclosure describes a burr hole device having an electrode. The burr hole device is configured to receive a catheter for delivering fluid to or withdrawing, shunting, or draining fluid from a brain of a subject and to receive electrical brain activity data via the electrode.

In embodiments, the burr hole device includes a body having a top surface and a bottom surface and defining a lumen extending from the top surface to the bottom surface. The body defines an upper flange portion and a lower portion. The lower portion is configured to be placed within the burr hole. The upper flange portion is configured to be disposed on the surface of the skull. The lumen is configured to receive a catheter configured to infuse fluid to, or withdraw fluid from, a brain of a subject. The burr hole device comprises an electrode disposed on the bottom surface of the lower portion.

The burr hole device may be configured such that the bottom surface of the lower portion is placed on a surface of a brain or above the surface of the brain when the burr hole device is implanted.

The burr hole device may have an electrical interconnect electrically coupled to the electrode. The electrical interconnect may be configured to electrically couple to an implantable medical lead.

An assembly may comprise the burr hole device and the catheter. The assembly may further comprise a sheath. The sheath may include a sheath body defining a sleeve lumen configured to receive the catheter, (ii) a recording electrode disposed on the sheath body, (iii) a cable, and (iv) a conductor electrically coupled to the recording electrode and extending in the cable. The burr hole device may comprise a groove configured to receive the cable. The catheter may be a therapeutic fluid delivery catheter, a catheter of a ventriculoperitoneal (VP) shunt, a catheter of an external ventricular drain (EVD), a catheter for aspirating CSF, or the like. The assembly may comprise a VP shunt, an EVD, an infusion device, which may be an implantable infusion device, configured to connect to the therapeutic fluid delivery catheter, or the like.

A method may include operatively coupling to signal apparatus the electrode disposed on the bottom surface of the lower portion of the body of the burr hole device.

A method may include disposing the burr hole device in or about a burr hole of a subject; inserting the catheter through the lumen of the burr hole device; and implanting a distal end of the catheter in a brain of the subject. The method may further include inserting the catheter through the sheath lumen; implanting the recording electrode of the sheath beneath the skull of the subject; and inserting the cable of the sheath in a groove of the burr hole device.

In an aspect, the present disclosure describes a burr hole device configured to receive a port device operatively couplable to a catheter for delivering fluid to, or withdrawing, shunting, or draining fluid from, a brain of a subject. The burr hole device is also configured to receive the catheter and comprises an electrode configured to detect brain electrical activity data.

In an aspect, the present disclosure describes an implantable sheath configured to receive a catheter and having an electrode configured to detect brain electrical activity data. The sheath and the catheter may be used with various embodiments of burr hole devices described herein.

In embodiments, an implantable sheath includes a body having a proximal end and a distal end and defining a lumen from the proximal end to the distal end. The lumen is configured to receive a catheter. The sheath comprises an electrode disposed on the body and a cable extending from the body in proximity to the proximal end. The sheath may comprise more than one electrode. One or more electrodes may be sensing electrodes. One or more electrodes may be referential electrodes. The sheath comprises a conductor electrically coupled to the electrode and extending in the cable. The cable comprises an electrical interconnect comprising a contact electrically coupled to the conductor. The sheath may be configured to be completely implanted in a subject.

An assembly may include the sheath, the catheter, and a burr hole device as described herein. The catheter may be a therapeutic fluid delivery catheter, a catheter of a ventriculoperitoneal (VP) shunt, a catheter of an external ventricular drain (EVD), a catheter for aspirating CSF, or the like. The assembly may comprise a VP shunt, an EVD, an infusion device, which may be an implantable infusion device, configured to connect to the therapeutic fluid delivery catheter, or the like.

In an aspect, the present disclosure describes a method for implanting a catheter and a sheath in a subject. The sheath may comprise (i) a sheath body defining a sleeve lumen configured to receive the catheter, (ii) one or more electrode, such as one or more recording or referential electrodes, disposed on the sheath body, (iii) a cable, and (iv) a conductor electrically coupled to the recording electrode and extending in the cable. When implanted a distal end of the catheter may be position in a brain of the subject and the electrode may be positioned in the brain.

In embodiments, the method comprises creating a first burr hole having a first diameter in a skull of a subject wherein the skull defines an edge of the first burr hole; and creating a second burr hole having a second diameter in skull. The second diameter is smaller than the first diameter. The first and second burr holes partially overlap. The first burr hole may be configured to receive a catheter or a burr hole device. The second burr hole may be configured to receive a cable of a sheath.

The method may further comprise creating a trench in the top surface of the skull. The trench may have a first end and a second end. The first end of the trench may intersect the second burr. The trench may extend away from the second burr hole towards the second end. The trench may have a first depth at the first end. The trench may have a second depth at the second end. The first depth may be greater than the second depth. The trench may be configured to receive a cable of a sheath.

The details of one or more aspects of the disclosure are set forth in the accompanying drawings and the description below. Other features, objects, and advantages of the techniques described in this disclosure will be apparent from the description and drawings, and from the claims.

### Definitions and Context for Defined Terms

All scientific and technical terms used herein have meanings commonly used in the art unless otherwise specified. The definitions provided herein are to facilitate understanding of certain terms used frequently herein and are not meant to limit the scope of the present disclosure.

As used herein, “disposed on” in the context of an electrode on a body, such as a sheath body refers to the electrode being associated with the body in a manner such that the position of the electrode maintains a position relative to the body and at least a portion of the electrode is exposed to tissue or fluid of a subject when the body is implanted in the subject. The electrode may be bonded, fastened, or adhered to the body, may be recessed into a portion of the body, or may be otherwise retained by the body, or the like.

As used herein, a “cable” is an assembly of one or more electrical conductors, such as wires, running along a length of the cable. The electrical conductors may be used to carry electric current. The conductors may be electrically insulated from one another. The conductors may be electrically insulated from the exterior of the cable by the material employed in forming the cable.

As used herein, “diameter” refers to the largest distance from one edge to another of an object along a section orthogonal to a longitudinal axis of the object. If the object is cylindrical, the diameter will be constant along the section. If the object is a rectangular prism having a square cross-section, the diameter is the distance from one corner of the square to the opposing corner of the square.

The terms “coupled” or “connected” refer to elements being attached to each other either directly (in direct contact with each other) or indirectly (having one or more elements between and attaching the two elements). Either term may be replaced to “couplable” or “connectable” to describe that the elements are configured to be coupled or connected. In addition, either term may be modified by “operatively” and “operably,” which may be used interchangeably, to describe that the coupling or connection is configured to allow the components to interact to carry out functionality.

As used herein, “treat,” “treatment,” or the like mean to reduce or alleviate one or more symptom or to slow the progression of the disease being treated.

As used herein, “intracranial” means within the skull of a subject. An intracranial electrode may be placed at any suitable location within the confines of a skull of a subject. For example, the intracranial electrode may be placed in the brain of the subject or on a surface of the brain of the subject. An intracranial electrode may be placed in brain parenchyma (“intraparenchymal”). An intracranial electrode may be placed within the cerebrum of a subject (“intracerebral”). An intracranial electrode may be placed within an intracranial blood vessel (“intravascular”). An intracranial electrode may be placed within a cerebral ventricle (“intraventricular”). iEEG signals include signals obtained from electrodes positioned on the surface of the brain of a subject and within the brain of the subject, including electrodes placed intraparenchymally, intracerebrally, intraventricular, and intravascularly.

As used herein, “withdrawing” fluid, such as cerebrospinal fluid (CSF), from a brain means removing the fluid from the brain. The removed fluid may be tested to determine one or more properties of the fluid, such as the concentration of one or more components of the removed fluid. The removed fluid, such as CSF, may be shunted to another location of the subject. The removed fluid may be drained from the subject. In some instances, the present disclosure discusses withdrawing, shunting, or draining CSF from the subject. In other instances, the present disclosure discusses withdrawing CSF from the subject without discussing shunting or draining. “Withdrawing” and “withdrawing, shunting, or draining” or the like, in the context of fluid relative to the brain, are used interchangeably herein.

As used herein, a “brain state” is a symptom or function of the brain that (i) involves multiple areas and neuronal networks of the brain and (ii) is reflected in brain activity. A brain state may be manifest in normal or abnormal brain activity. Motor function, speech function, visual function, somatosensory sensation function, smell function, and the like, in and of themselves, are not brain states. These functions, in and of themselves, impact smaller, more discrete, less distributed regions of the brain. For example, visual function involves the visual cortex, visual radiations, and optic tract. A brain state may, however, involve one or more of motor function, speech function, visual function, smell function, and the like. A brain state may involve activity in 2 or more Brodmann areas, 3 or more Brodmann areas, 4 or more Brodmann areas, 5 or more Brodmann areas, 6 or more Brodmann areas, 7 or more Brodmann areas, 8 or more Brodmann areas, 9 or more Brodmann areas, or 10 or more Brodmann areas.

As used herein, a “psychological brain state” is a brain state having a mental or emotional component. A subject is typically aware of their psychological brain state in their feelings and thoughts. Examples of psychological brain states include general affect or mood, anxiety, depression, addiction, obsession, suicidal thoughts, hallucinations, cognition, attention, post-traumatic stress, and the like, and degrees thereof. As an example, a traumatic memory may involve one area of the brain, but post-traumatic stress disorder (PTSD) is the impact of that memory which distracts attention, causes fear, and/or impacts cognitive function. Accordingly, PTSD involves or impacts multiple regions and neural networks of the brain.

Psychological brain states do not include seizure activity and/or motor activity alone. However, electrical brain activity associated with epileptic and/or motor activity may be relevant to a broader psychological brain state.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, singular forms “a,” “an” and “the” include plural referents unless the context clearly dictates otherwise.

As used herein, the term “or” is generally employed in its sense including “and/or” unless the content clearly dictates otherwise. The term “and/or” means one or all the listed elements or a combination of any two or more of the listed elements. The use of and/or” in some locations of the present disclosure is not intended to mean that the use of “or” in other locations cannot be interpreted as “and/or.”

The phrases “at least one of” and “one or more of” followed by a list refers to any one of the items in the list and any combination of two or more items in the list.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

Any direction referred to herein, such as “top,” “bottom,” “side,” “upper,” “lower,” and other directions or orientations are described herein for clarity and brevity but are not intended to be limiting of an actual device or system. Devices and systems described herein may be used in a number of directions and orientations.

As used herein, “providing” an article, device, or system means manufacturing the article, device, or system, assembling the article, device, or system, purchasing the article, device, or system, or otherwise obtaining the article, device, or system.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must).

The words “include,” “including,” and “includes” indicate open-ended relationships and therefore mean including, but not limited to. Similarly, the words “have,” “having,” and “has” also indicated open-ended relationships, and thus mean having, but not limited to. Similarly, the terms “comprise” and “comprising” indicate open-ended relationships, and thus mean comprising, but not limited to. The terms “consisting essentially of” and “consisting of” are subsumed within the term “comprising.” For example, a catheter comprising tubing may be a catheter consisting of tubing. The term “consisting essentially of” means a recited list of one or more items belonging to an article, kit, system, or method and other non-listed items that do not materially affect the properties of the article, kit, system, or method.

The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a catheter connector may be configured to place a lumen of a catheter in fluid communication with a fluid path, even when the catheter is not connected to the catheter connector).

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

### BRIEF DESCRIPTION OF THE DRAWINGS

FIGS. 1-3 are a schematic perspective views of an embodiment of a burr hole device.

FIG. 4A is a schematic side view of the burr hole device of FIGS. 1-3.

FIGS. 4B and 4C are side views of alternative embodiments of burr hole devices.

FIGS. 5-7 are schematic top views of embodiments of burr hole devices.

FIG. 8 is a schematic bottom plan view of an embodiment of a burr hole device having an electrode.

FIGS. 9-10 are side plan views of embodiments of the device shown in FIG. 8.

FIG. 11 is a schematic sectional view of an embodiment of a burr hole device configured to receive a cranial port device.

FIG. 12 is a schematic perspective sectional view of the burr hole device of FIG. 11.

FIG. 13 is a bottom plan view of the burr hole device of FIG. 11.

FIG. 14 is a top plan view of the burr hole device of FIG. 11.

FIG. 15 is a schematic side sectional/block view of a burr hole device comprising electronic components.

FIGS. 16A-D are side views (A and C), a sectional view (B), and a top view (D) of an embodiment of a burr hole device.

FIG. 17 is a schematic side view of an embodiment of the sheath containing electrodes.

FIG. 18 is a schematic side view of an embodiment of an assembly comprising a cranial port, the sheath of FIG. 17, and a catheter relative to a skull.

FIG. 19 is a schematic side view of the assembly of FIG. 18 with the skull removed.

FIG. 20 is a schematic cross-sectional view of the assembly of FIG. 18 relative to a bottom surface of the skull.

FIG. 21 is a schematic perspective view of the assembly of FIG. 18.

FIG. 22 is a schematic perspective view of the assembly of FIG. 21 with the skull removed.

FIG. 23 is a schematic perspective view of the assembly of FIG. 18.

FIG. 24 is a schematic side view of the assembly of FIG. 18.

FIG. 25 is a schematic perspective view of an embodiment of an assembly comprising a cranial port device, a sheath, and a catheter.

FIG. 26 is a schematic side view of the assembly of FIG. 25.

FIG. 27 is a schematic view of an assembly comprising a cranial port device, a sheath, and a catheter implanted relative to a head of a subject.

FIG. 28 is a schematic view of a system comprising the assembly of FIG. 27 and signal apparatus implanted relative to the head of the subject.

FIGS. 29 and 30 are schematic top view portions of skulls having first and second burr holes therethrough where the burr holes are created according to methods described herein.

FIG. 31 is a schematic perspective view of a portion of the skull shown in FIG. 30.

While the invention is susceptible to various modifications and alternative forms, specific embodiments thereof are shown by way of example in the drawings and may herein be described in detail. The schematic drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

Features or elements shown in a given figure that are not shown in another figure may be present in the other figure in which the features or elements are not shown. That is, the features and elements shown in the figures are interchangeable. For example, an embodiment of a burr hole device that is shown in the figures without through openings configured to receive screws, an electrical interconnect, a cable, or any other feature or element may, in other embodiments, include through openings, an electrical interconnect, a cable, or any other feature or element illustrated in other figures.

### DETAILED DESCRIPTION

The present disclosure relates to, among other things, devices and systems configured for use with a catheter for delivering therapeutic agents to the brain or withdrawing CSF from the brain. The devices and systems may include a sheath configured to receive the catheter. The sheath may include an electrode configured to be intracranially positioned for recording brain activity data. The devices and systems may include a burr hole apparatus configured to receive the catheter and the sheath or portions thereof.

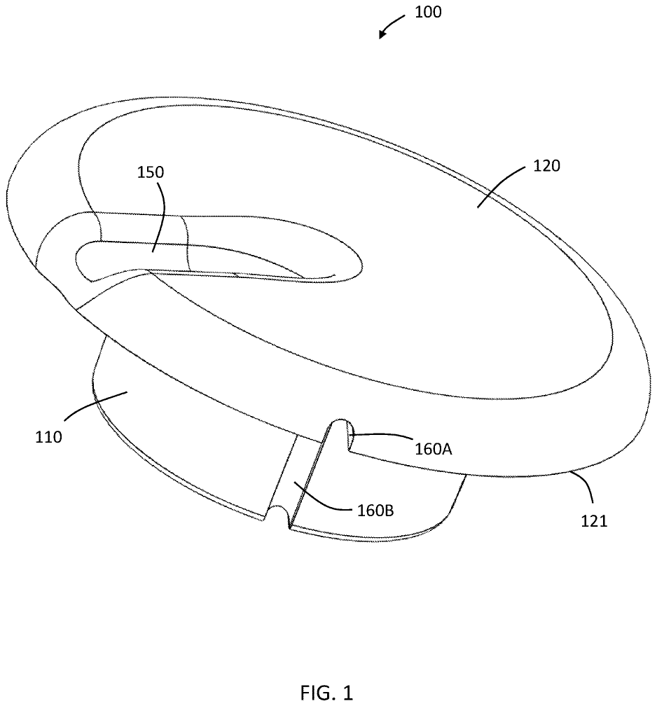

FIGS. 1, 2, 3, and 4A show an embodiment of a burr hole device 100 configured for use with a catheter for delivering fluid to or removing fluid from a brain of a subject. The burr hole device 100 is also configured to receive a cable. The cable may comprise a conductor electrically coupled to an electrode. The electrode may be disposed on the catheter, a lead, or a sheath configured to receive the catheter.

The burr hole device 100 has a body 110 having a top surface 120 and a bottom surface 130A, 130B and defining a lumen 140 extending from the top surface 120 to the bottom surface 130. The lumen 140 is configured to receive a catheter configured to allow fluid to flow to or from a brain of a subject. For example, the catheter may be configured to deliver fluid therapeutic or diagnostic compositions to the brain of the subject, may be configured to permit aspiration of cerebral spinal fluid (CSF) from the brain of the subject, may be configured to drain or shunt CSF from the brain of the subject, or the like.

The body 110 defines a top groove 150 along the top surface 120. The top groove 150 extends from the lumen 140 towards a lateral edge 121 of the top surface 120 of the body 110. The top groove 150 is configured to receive the catheter as the catheter exits the lumen 140. The top groove 150 may be configured to snuggly or grippingly engage the catheter to retain the catheter relative to the burr hole device 100. Alternatively, the top groove may be configured to loosely receive the catheter so that the catheter may be readily removed from the brain by pulling on a proximal portion of the catheter at a distance from the burr hole device 100. Such ease of removal may be beneficial for certain catheters where explant is routine, such as with catheters that are part of an external ventricular drain (EVD) apparatus. Often, EVD catheters exit the skin of the subject at a distance from the burr hole. Explanting the EVD catheter by pulling on an externalized portion of the catheter may be more desirable than reopening the scalp in proximity to the burr hole to remove the catheter.

The body 110 of the burr hole device 100 depicted in FIGS. 1-4 defines an upper flange portion 125 and a lower portion 170. The lower portion 170 is configured to be placed within the burr hole. Preferably, the lower portion 170 of the body 110 has a clearance of 1 millimeter or less when disposed in the burr hole to help stabilize the device 100 when implanted. In embodiments, the lower portion 170 of the body 110 has an outer diametric dimension ODL in a range from 3 millimeters to 20 millimeters, which outer diametric dimension may depend on the size of the burr hole. In embodiments, the lower portion 170 of the body 110 has an outer diametric dimension ODL in a range from 10 millimeters to 20 millimeters. In embodiments, the lower portion 170 of the body 110 has an outer diametric dimension ODL in a range from 5 millimeters to 14 millimeters, such as from about 11 millimeters to about 13 millimeters. Typically, the outer surface of the lower portion 170 is generally cylindrical as depicted in FIGS. 1, 2, 3, and 4A or frustoconical, and the lower portion 170 of the device 100 may have a generally circular cross-section.

When the burr hole device 100 is implanted, a bottom surface 130B of the lower portion 170 of the device 100 preferably does not extend substantially beyond the bottom of skull when received in the burr hole. Accordingly, the height of lower portion 170 may vary depending on the thickness of the skull of the subject into which the burr hole device 100 is implanted. As an example, a thickness of a human adult skull may typically be in a range from about 6.5 millimeters to about 7 millimeters.

In some embodiments, the height of the lower portion 170 (HL) of the burr hole device 100 (distance from the bottom surface 130A of the upper flange portion 125 to the bottom surface 130B of the lower portion 170) is in a range from about 3 millimeters to about 7 millimeters. For example, the height of the lower portion 170 may be in a range from about 4 millimeters to about 6 millimeters or from about 4.5 millimeters to about 5.5 millimeters.

The upper flange portion 125 is configured to be disposed on the surface of the skull adjacent to the burr hole. That is, the bottom surface 130A of the upper flange portion 125 may rest on the skull when the device 100 is implanted. The upper flange portion 125 has an outer diametric dimension ODu that is greater than the diameter of the burr hole. In embodiments, the outer diametric dimension ODu of the upper flange portion 170 is in a range from 15 millimeters to 30 millimeters.

The bottom surface 130A of the upper flange portion 125 defines a bottom groove 160A configured to receive a cable comprising a conductor electrically coupled to an electrode configured to be implanted in the brain of the subject. The bottom groove 160A extends from a lateral edge 121 of the bottom surface 130A towards the lumen 140. The cable may be protected by the bottom groove 160A when implanted, rather than being pinched between the upper surface of the skull and the bottom surface 130A of the upper flange portion 125.

The lower portion 170 of the device 100 may define a side groove 160B that may run the length of the lower portion 170. The side grove 160B intersects with and meets bottom groove 160A. The side groove 160B is configured to receive the cable such that the cable. The cable may be positioned in the side groove 160B between the side of the burr hole and the body 110 of the lower portion 170 when the burr hole device 100 is implanted. When clearances are small between the lower portion 170 and the burr hole, the cable may be protected in the side groove 160B, as opposed to being pinched between the lower portion 170 of the device 100 and the side of the burr hole.

The burr hole device 100 may optionally comprise bottom groove 160C as depicted in FIGS. 1, 2, 3, and 4A. Bottom groove 160C extends from the lumen 140 to a lateral edge of the bottom surface 130B of the lower portion 170 of the burr hole device 170. Bottom groove 160C may intersect and meet with side groove 160B.

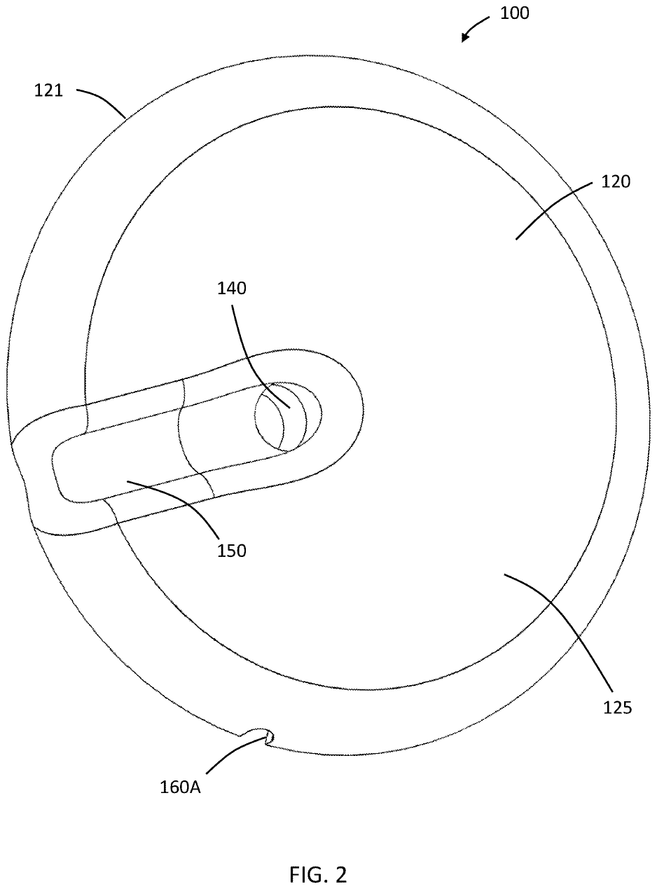

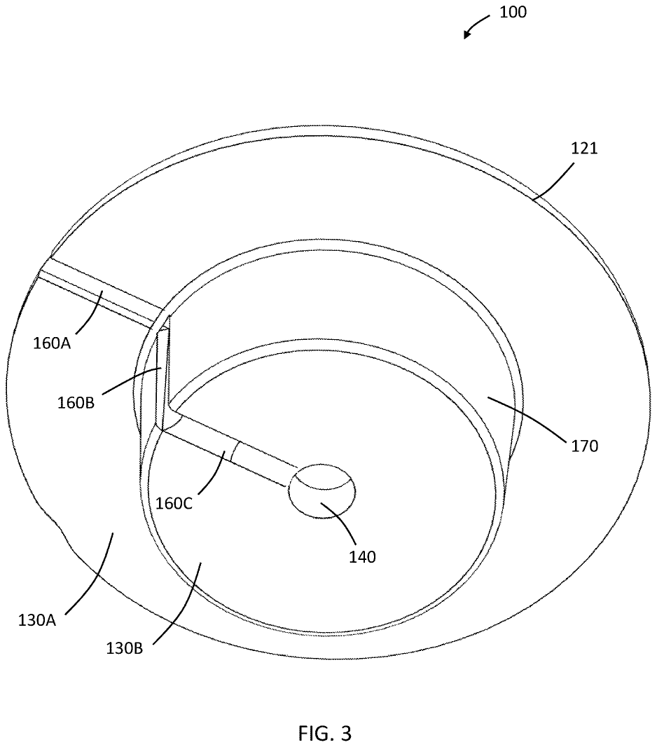

In embodiments, a burr hole device 100 lacks a lower portion 170 or has a lower portion 170 with having a small height, as depicted in FIGS. 4B and 4C. The height of the lower portion in FIG. 4B may range from about 0.1 millimeters to about 1 millimeter. The lower portion 170 may be inserted into a burr hole to assist with aligning the top upper portion 125 relative to the burr hole. The upper flange portion 125 may include a groove 150 for receiving a portion of a catheter.

When the burr hole device 100 lacks a lower portion as shown in FIG. 4C, the burr hole device may be visually aligned over the burr hole.

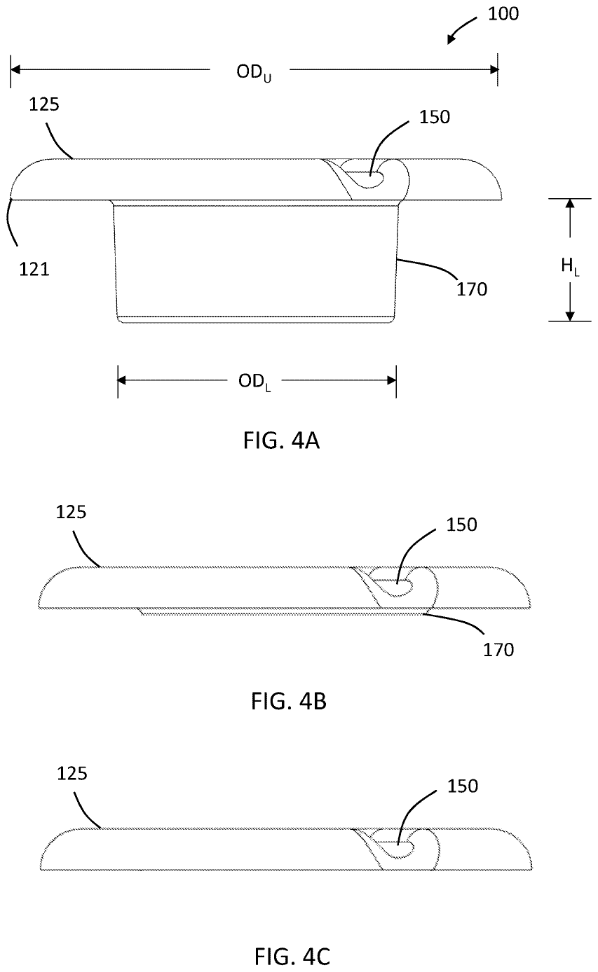

FIGS. 5-7 show embodiments of burr hole devices 100 having a groove 165 configured to receive a cable, such as a cable of a sheath, on the top surface 120 of the upper flange portion 125. The burr hole devices 100 of FIGS. 5-7 may, otherwise, be similar to the burr hole devices 100 shown in FIGS. 1-3 and 4A-C. The burr hole devices 100 of FIGS. 5-7 may have some or all features or elements of the burr hole devices 100 of FIGS. 1-3 and 4A-C. For example, the devices 100 of FIGS. 5-7 may comprise a bottom groove 160A (or a bottom grooves 160A, 160C) or a bottom groove 160A (or a bottom grooves 160A, 160C) and a side grove 160B as shown in FIGS. 1-4. By having grooves for a cable on the bottom surface 130A (or 130A and 130B) and the top surface 120, the burr hole device 100 may be used with a variety of different configurations of cables, such as cables of sheaths. Having grooves for a cable on the bottom surface 130A (or 130A and 130B) and the top surface 120, the burr hole device 100 may also provide the implanting physician with options for different routes for cable feedthrough on a case-by-case basis.

As shown in FIG. 5, the top surface 120 of the upper flange portion 125 of the burr hole device 100 defines a first top groove 150 configured to receive a catheter and a second top groove 165 configured to receive a cable. The first 150 and second 165 top grooves extend from the lumen 140 to a lateral edge 121 of the top surface 120. A portion of the catheter and a portion of the cable may exit the lumen 140 and may be placed in the respective top groove 150, 165. The first top grove 150 may be configured to snuggly or grippingly receive the catheter or may be configured to loosely receive the catheter. The second top groove 165 may be configured to snuggly or grippingly receive the cable or may be configured to loosely receive the cable.

While the first top groove 150 and second top groove 165 shown in FIG. 5 extend in generally opposite directions from the lumen 140 across the top surface 120, they may extend in any suitable orientation relative to one another.

As shown in FIG. 6, the top surface 120 of the upper flange portion 125 of the burr hole device 100 defines a top groove 150 extending from the lumen 140 towards a lateral edge 121 of the top surface 120. The top groove 150 comprises a lower groove portion 165′ and an upper groove portion 150′. The lower groove portion 165′ is configured to receive a cable and has a smaller diametric dimension than the upper groove portion 150′, which is configured to receive a catheter. The upper groove portion 150′ is configured to receive the catheter such that the catheter is positioned over the cable when the both the cable and the catheter are received in the top groove 150. A portion of the catheter and a portion of the cable may exit the lumen 140 and may be placed in the respective top groove portion 150′, 165′. The upper top grove portion 150′ may be configured to snuggly or grippingly receive the catheter or may be configured to loosely receive the catheter. The lower top groove portion 165′ may be configured to snuggly or grippingly receive the cable or may be configured to loosely receive the cable.

In FIG. 7, the burr hole device 100 has a first lumen 140 extending through the body from the top surface 120 to the bottom surface (e.g., 103B as shown in FIGS. 1-4). The burr hole device 100 has a second lumen 145 extending through the body from the top surface 120 to the bottom surface. The first 140 and second 145 lumens partially overlap. The first top groove 150, which is configured to receive a catheter, extends from the first lumen 140 to a lateral edge 121 of the upper surface 120 of the body 110. The second top groove 165, which is configured to receive a cable, extends from the second lumen 145 to a lateral edge 121 of the upper surface 120 of the body 110. The diameter of the second lumen 145 is smaller than the diameter of the first lumen 140. The first lumen 140 is configured to receive the catheter. The second lumen 145 is configured to receive the cable.

While the first top groove 150 and second top groove 165 shown in FIG. 5 extend in generally opposite directions from the lumen 140 across the top surface 120, they may extend in any suitable orientation relative to one another.

It should be understood that the burr hole device 100 shown in FIG. 6 may be readily modified to include a second lumen 145 (and the lower grove portion 165′ may extend from the second lumen 145) as shown in FIG. 7.

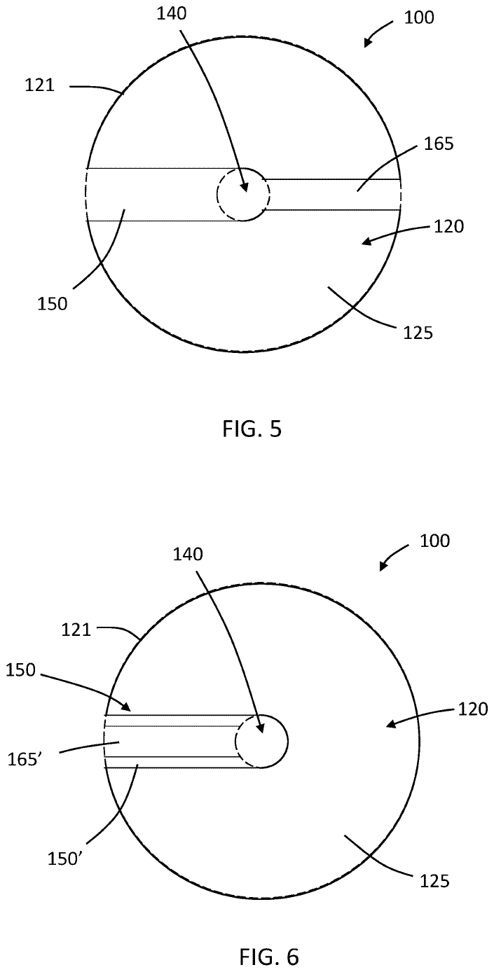

FIGS. 8-10 show embodiments of burr hole devices 100 having an electrode 180. The electrode 180 may be configured to detect brain electrical activity, such as EEG signals or to act as a return path for electrical sensing with other electrodes. The burr hole devices 100 of FIGS. 8-10 may, otherwise, be similar to the burr hole device 100 shown in FIGS. 1-7. The burr hole devices 100 of FIGS. 8-10 may have some or all features or elements of the burr hole device 100 of FIGS. 1-7.

The electrode 180 is disposed on or may be in proximity to the bottom surface 130B of the lower portion 170 of the burr hole device 100. Disposing the electrode 180 on or in proximity to the bottom surface 130B may place the electrode 180 on or in proximity to a surface of the brain when the lower portion 170 of the burr hole device 100 is positioned in the burr hole.

In embodiments, the electrode 180 is disposed on at least a portion of the bottom surface 130B of the burr hole device 100. The electrode 180 may be annular as shown in FIG. 8 or may have any other suitable shape. The electrode may extend over the entire bottom surface 130B or over a portion of the bottom surface 130B of the device 100. More than one electrode (not shown) may be disposed on the bottom surface 130B of the device 100.

The electrode 180 may serve as a ground electrode, reference electrode, or working electrode, or combinations thereof. The electrode 180 may be operatively couplable with signal apparatus that processes signals from the electrode 180 and other electrodes.

The electrode 180 may be made from any suitable material. Suitable materials for implantable electrodes are well-known to those of skill in the art. Materials suitable for deep brain stimulation electrodes are suitable materials for electrodes of the catheters described herein. In embodiments, the electrode 180 is made from platinum or a platinum iridium alloy.

The electrode 180 may have any suitable thickness. For example, the electrode 180 may have a thickness from about 100 microns to about 3 millimeters, such as from about 200 microns to about 2 millimeters. The electrode 180 may be formed from a foil.

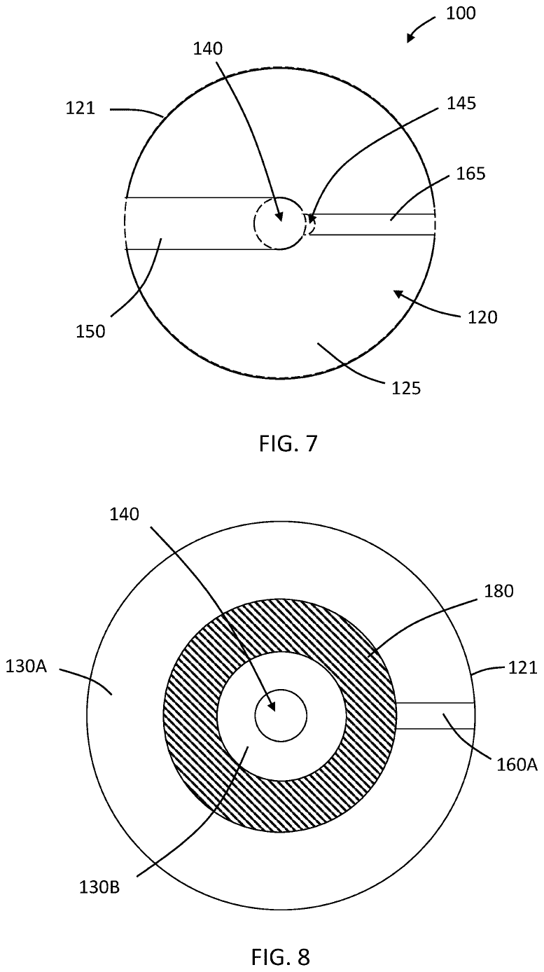

As shown in FIG. 9, the burr hole device 100 may include an electrical interconnect 190 electrically coupled to the electrode 180. A conductor may run through the body of the burr hole device 100 from the electrode 180 to a contact of the electrical interconnect 190. The electrical interconnect 190 may be configured to electrically couple with an implantable medical lead or suitable wire for carrying electrical signals from the burr hole device 100 to signal apparatus for processing or transmitting signals received by the electrode 180.

As shown in FIG. 10, the burr hole device 100 may comprise a cable 195 electrically coupled to the electrode 180. The cable 195 may comprise a conductor electrically coupled to the electrode 180. The cable 195 may comprise a contact (not shown) for electrical connection to, for example, signal apparatus.

The burr hole device 100 having an electrode 180 may comprise a bottom groove 160A on the bottom surface 130A of the upper flange portion 125, and may also comprise a side groove (e.g., 160B as shown in the device of FIGS. 1-4) along the side of the lower portion 170 that meets with the bottom groove 160A. The bottom groove 160A and side groove, if present, may receive a cable. The cable may comprise a conductor electrically coupled to the electrode 180.

In embodiments, the bottom groove 160A and side groove, if present, receive a cable comprising a conductor electrically coupled to an electrode disposed on the catheter or a sheath.

The burr hole device 100 may comprise an electrical interconnect 190 or a cable 195 and may comprise a bottom groove 160A and optional side groove. Accordingly, the burr hole device 100 is configured to allow signals from the electrode 180 of the burr hole device 100 and electrodes of other devices, such as a catheter or sheath, used with the burr hole device 100 to be delivered to, for example, signal apparatus. Signals from electrodes of the associated devices may be carried through a cable received in the bottom groove 160A and optional side groove, and signals from the electrode 180 of the burr hole device 100 may be carried through the cable 195 or electrical interconnect 190.

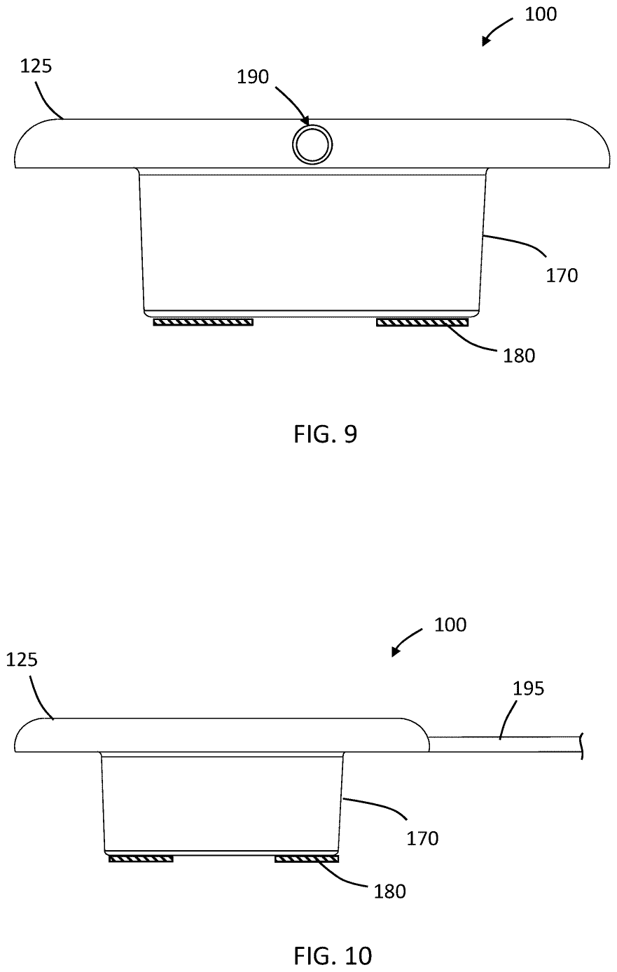

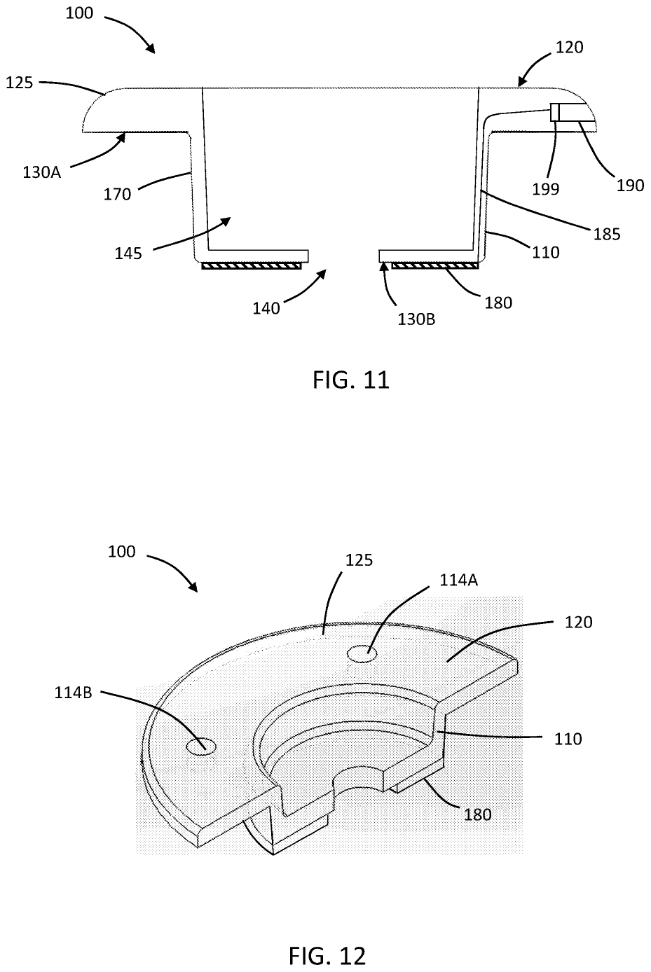

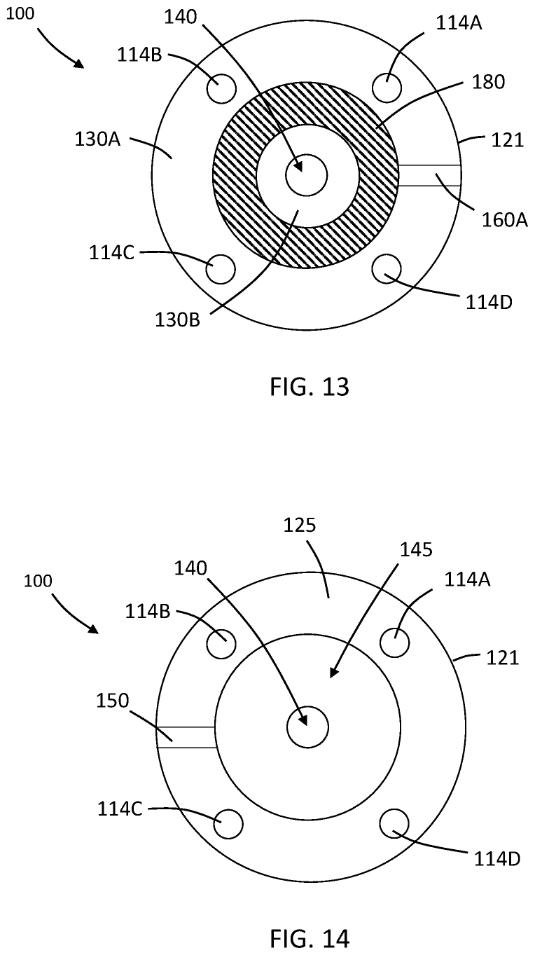

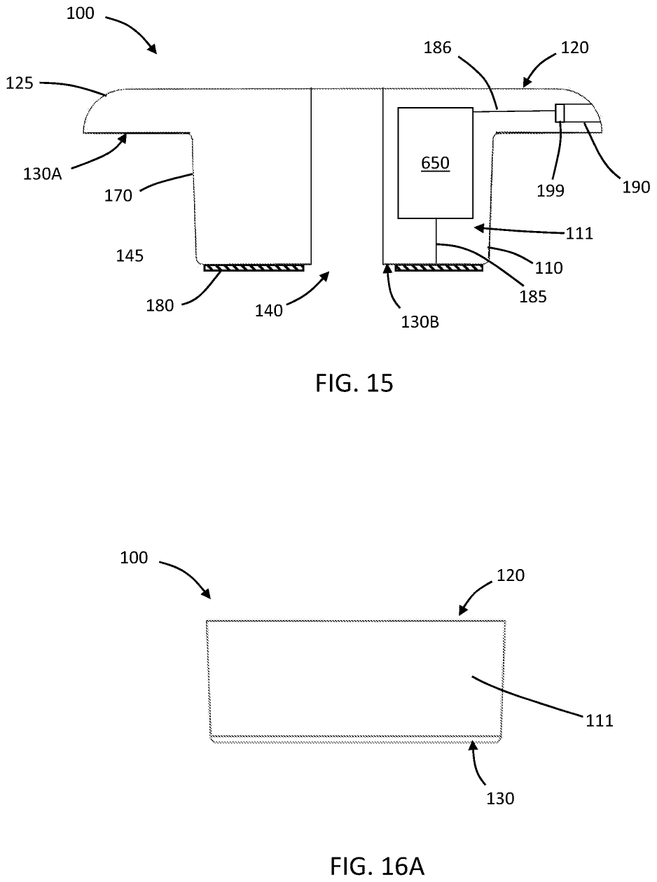

FIGS. 11-14 show an embodiment of a burr hole device 100 configured to receive a cranial port device. The burr hole device 100 of FIGS. 11-14 may, otherwise, be similar to the burr hole device 100 shown in FIGS. 1-10. The burr hole devices 100 of FIGS. 11-14 may have some or all features or elements of the burr hole device 100 of FIGS. 1-10.

The burr hole device 100 may be configured to receive any suitable cranial port device. For example, the burr hole device 100 may receive a ventriculoperitoneal shunt valve housing, an Ommaya reservoir, a cranial port device as described in, for example, U.S. 2022/016338-A1, entitled IMPLANTABLE CRANIAL MEDICAL DEVICE, filed on Jul. 15, 2021, and published on Jan. 20, 2022, which published patent application is hereby incorporated herein in its entirety by reference to the extent that it does not conflict with the disclosure presented herein, or the like.

The burr hole device 100 of FIGS. 11-14 includes a body 110 defining an upper flange portion 125 and a lower portion 170. The lower portion 170 is configured to be placed within the burr hole and the upper flange portion 125 is configured to be disposed on the surface of the skull. The body 110 defines a cavity 145 for receiving a cranial port device. The lower portion 170 defines an opening 140 in communication with the cavity 145. The opening 140 is configured such that a catheter or a catheter connector of the cranial port device may be inserted therethrough. The cavity 145 may be shaped similarly to an outer surface of a housing of the cranial port device.

The upper flange portion 125 defines a top groove 150 that extends from the cavity 145 towards a lateral edge 121 of the top surface 120. The top groove 150 is configured to receive a catheter, such as a catheter coupled to the cranial port device, if any.

The bottom surface 130A of the upper flange portion 125 defines a groove 160A. The device 100 may also comprise a side groove (e.g., 160B as shown in the device of FIGS. 1-4) along the side of the lower portion 170 that meets with the bottom groove 160A. The bottom groove 160A and side groove, if present, may receive a cable comprising a conductor electrically coupled to an electrode disposed on a catheter or a sheath.

The burr hole device 100 of FIGS. 11-14 includes an electrode 180 disposed on the bottom surface 130B of the lower portion 170. The device 100 may include an electrical interconnect 190 comprising a contact 199 for electrically coupling to a lead or other suitable cable to electrically couple the electrode 180 to, for example, signal apparatus. The device 100 includes a conductor 185 that runs through the body 110 of the device 100 and electrically couples the electrode 180 to the contact 199 of the electrical interconnect 190. Alternatively, the burr hole device 100 may comprise a cable (e.g., 195 as shown in FIG. 10) for carrying signals from the electrode 180 to, for example, signal apparatus.

The burr hole device 100 may include one or more through holes 114A-D (four shown) through the upper flange portion 125. The through holes 114A-D may be configured to receive screws for securing the burr hole device 100 to the skull of the subject. While not shown in FIGS. 1-10, it will be understood that the burr hole devices of FIGS. 1-10 may also have through holes.

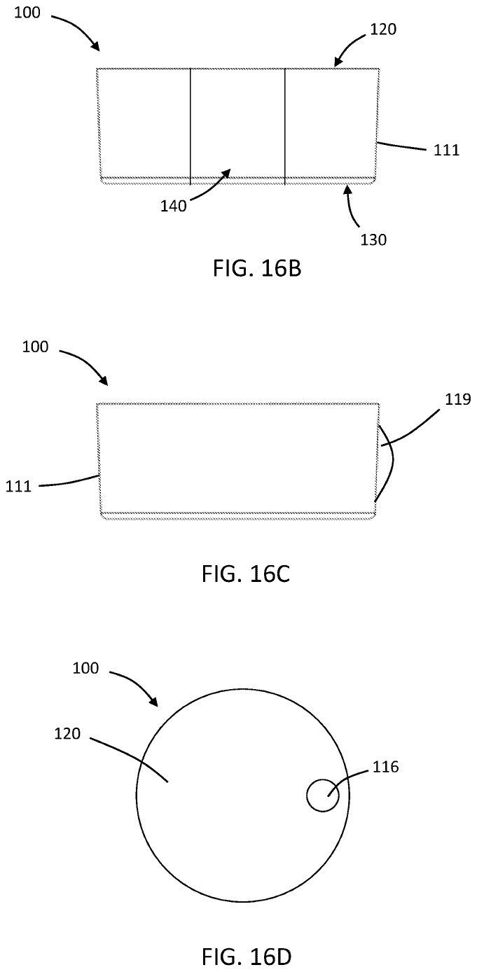

The burr hole device 100 of FIG. 15 is similar to the burr hole device of FIG. 11, except that the burr hole device 100 of FIG. 15 includes one or more electronic component 650 operably coupled to the electrode 180 or electrodes via one or more conductors 185. The electrode 180 or electrodes may be positioned at a bottom surface 130B of the lower portion 110 of the burr hole device 100. The one or more electronic components 650 may be housed in a cavity 111 defined by a body of the lower portion 110 of the device. The electronic components 650 may be operatively coupled to electrodes of another device or devices, such as a sheath, a lead, or the like.

The one or more electronic component 650 may one or more of: store, transmit, and process signals from the electrode 180 or electrodes. The one or more electronic component 650 may include one or more of a power source, a transmitter for transmitting brain activity obtained from the electrodes, a controller for controlling other components, a processor, memory for storing brain activity data, which may or may not be processes, and memory containing instructions carried out by the controller or processor. The power supply may comprise a battery. The battery may be rechargeable, wireless powered, or rechargeable and wirelessly powered. The signal apparatus may comprise an inductive coil, solenoid, or other suitable components configured to permit wireless powering or charging by an external apparatus and to transmit data regarding the signals recorded by the electrodes to the external apparatus.

The external device may be placed in proximity to the burr hole device 100 to permit wireless charging, powering, and/or data transfer. Any suitable external device may be used. In embodiments, the external device may be worn by the subject. For example, the external device may comprise a hat having electronic components that may be placed in proximity to the burr hole device 100 when the subject wears the hat. The external device may be continuously placed in proximity to the burr hole device 100 to permit transmission of brain activity data from the one or more electronic components 650 to be transmitted to the external device. The external device may be worn for short periods of time or for long durations. When the external device is not in proximity to the burr hole device the one or more electronic components 650 the one or more electronic components 650 of the burr hole device may store brain activity data. Once the external device is placed in proximity to the burr hole device 100, the one or more electronic components 650 may transmit the stored data to the external device.

The electrical components 650 may optionally be electrically coupled to one or more contact 199 of an electrical interconnect 190 via one or more conductors 186. The electrical interconnect 190 may be used to connect the control electronics 650 to other signal apparatus, which may be implanted.

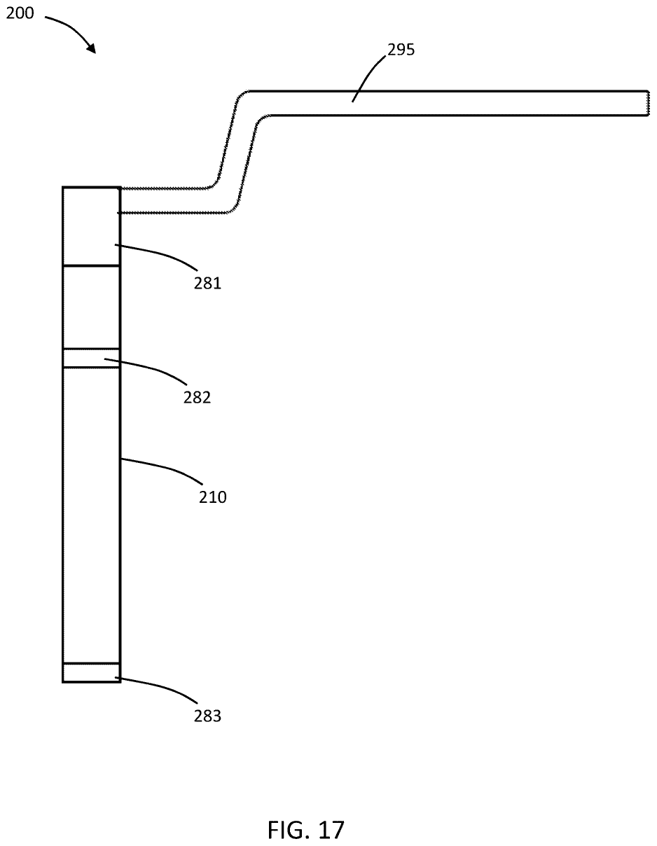

Referring now to FIGS. 16A-D, an embodiment of a burr hole device 100 configured to be inserted into a burr hole of a subject and to be secured against a side of the burr hole is shown. The depicted burr hole device 100 does not contain an upper flange portion. However, embodiments of burr hole devices having an upper flange portion (e.g., as shown in, and described regarding, FIGS. 1-14) and configured to be inserted into a burr hole of a subject and to be secured against a side of the burr hole are contemplated herein.

The burr hole device in FIGS. 16A-D has a body 111 defining a top surface 120, a bottom surface 130 and a lumen 140 extending through the body 111 from the top surface 120 to the bottom surface 130. The lumen 140 may be configured to receive one or more of a catheter, a cable (such as a cable of a sheath), a lead, or the like. The catheter may be configured to infuse fluid to, or withdraw fluid from, a brain of a subject. The catheter may be a therapeutic fluid delivery catheter, a catheter of a ventriculoperitoneal shunt, a catheter of an external ventricular drain, a catheter for aspirating CSF, or the like.

The body 111 may define a cavity for housing one or more electronic components, such as electronic components 650 as depicted in FIG. 15. The device 100 may comprise an electrode, such as an electrode 180 as depicted in FIG. 15.

At least a portion of the body 111 of the burr hole device 100 of FIGS. 16A-D is configured to be inserted into a burr hole. In embodiments, the entire device 100 is configured to be received in the burr hole. The burr hole device 100 comprises an expansion member 119 that may be deployed when the body 111 is in the burr hole. The expansion member 119 is configured to expand against a side of the burr hole to anchor the body 111 within the burr hole. The expansion member 119 is deployable from a retracted state (FIG. 16A) to an expanded state (FIG. 16C). When the expansion member 119 is in the retracted state, the body 111 is slidably receivable in the burr hole. The expansion member 119 may be operatively coupled to a user actuatable member 116 that, when actuated, causes the expansion member 119 to adapt the expanded state. The user actuatable member 116 may be a screw, bolt, or the like, that may be turned by a user when using an appropriate tool, such as a screwdriver, or the like.

Any suitable expansion member 119 and user actuatable member 116 or combination thereof may be employed. For example, the user actuatable member 116 may comprise a screw and expansion member 119 may be an anchor that expands as the screw is advanced. The user actuatable member 116 may comprise a bolt and expansion member 119 may comprise an elongate member. The elongate member may be coupled to the bolt such that rotation of the bolt causes rotation of rotation of an elongate member about an axis of the bolt to cause the elongate member press against the side of the burr hole to retain the body 111 in the burr hole.

The body of the burr hole devices described herein may be made from any suitable material or materials. In embodiments, the body of the burr hole device comprises rigid materials such as a hard plastic, ceramic, glass, or metallic material, or a combination thereof. Rigid material may have a higher durometer than more flexible materials. Examples of materials that may be used to form the body of the burr hole device include one or more of a high-performance thermoplastic or relatively rigid plastic material, such as polyurethane, polycarbonate, polysulfone, polyether ether ketone (PEEK), nylon, and Ultra High Molecular Weight Polyethylene (UHMWPE); and a biocompatible metal, such as a stainless steel alloy, titanium, and nitinol. Preferably, the material is compatible with magnetic resonance imaging (MRI). Preferably, the upper flange comprises a biocompatible material or comprises an exterior biocompatible coating.

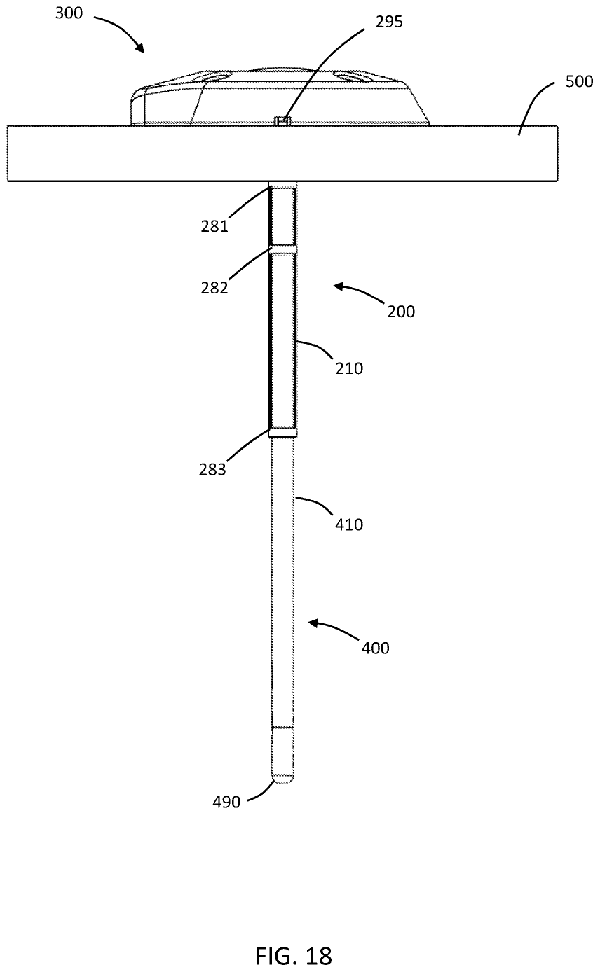

Referring now to FIG. 17, an embodiment of a sheath 200 is shown. The sheath 200 is configured to receive a catheter via a lumen that runs through the length of the body 210 of the sheath 200. The sheath 200 comprises one or more electrodes 281, 282, 283 (three shown) and a cable 295 comprising one or more conductors. Each conductor may be independently electrically coupled to an electrode 281, 282, 283. The cable 295 may comprise contacts (not shown), each independently electrically coupled to a conductor such that the conductors discretely electrically couple the contacts to the electrodes 281, 282, 283.

The sheath 200 is configured to be implanted such that the one or more electrodes 281, 282, 283 are in contact with the brain of a subject. The one or more electrodes 281, 282, 283 may be used to detect brain activity, such as electrocochleogram (EEG) activity. Contacts of the cable 295 or an interconnect electrically coupled with the cable 295 may be used for electrical coupling to signal apparatus.

The sheath 200 may be used with a burr hole device 100 as described herein, such as a burr hole device as shown in FIGS. 1-16 or may be used without a burr hole device as described herein. The cable 295 may be received within a groove of the burr hole device 100, such as in groove 160A, 160B, 160C, or 165, or groove portion 165′ shown in FIGS. 1-16. If the burr hole device 100 includes an electrode 180, the electrode 180 of the burr hole device 100 and the one or more electrodes 281, 282, 283 of the sheath 200 may be operatively coupled to signal apparatus so that the signals detected by the electrode 180 of the burr hole device 100 and the one or more electrodes 281, 282, 283 of the sheath 200 may be appropriately processed or transmitted.

The diameter of the lumen defined by the sheath body 210 is preferably no more than 1 mm greater than the outer diameter of the catheter to keep minimal the outer diameter of the sheath body 210. In embodiments, the diameter of the lumen is no more than 0.9 mm, no more than 0.8 mm, no more than 0.7 mm, no more than 0.6 mm greater, no more than 0.5 mm greater, no more than 0.4 mm greater, no more than 0.3 mm greater, no more than 0.2 mm greater, or no more than 0.1 mm greater than the outer diameter of the catheter. The diameter of the lumen may be substantially the same (e.g., within 10%) along the length of the sheath body 210 or may vary along the length of the sheath body 210.

In embodiments, the diameter of the lumen of the sheath body 210 is from about 2 mm to about 5 mm, such as from about 2.2 mm to about 4 mm, or from about 2.5 mm to about 3.5 mm.

The material of the sheath body 210 defining the lumen and the material defining an outer surface of the catheter preferably have a low coefficient of friction to allow the catheter to easily slide through the lumen. Preferably, the lumen of the sheath body 210 comprises a smooth surface. The surface of the sheath body 210 that defines the lumen may be lubricious or a lubricious coating may be applied to the surface of the lumen.

The sheath body 210 preferably is preferably sufficiently rigid to permit the sheath body 210 to be advanced through brain parenchymal tissue. The sheath body 210 is preferably sufficiently flexible to permit movement along with movement of the brain so that the sheath body 210 does not damage brain tissue when implanted.

One of skill in the art will understand that the rigidity and flexibility of the sheath body 210 will depend on the material from which the elongate body is formed as well as the thickness of the sheath body 210. The sheath body 210 may have any suitable thickness from an outer surface to the lumen. In embodiments, the sheath body 210 has a thickness from about 0.1 mm to about 1 mm, such as from about 0.2 mm to about 0.5 mm.

The sheath body 210 may be formed from any suitable material. For example, the elongate body may be formed from polydimethylsiloxane (PDMS).

The electrodes 281, 282, 283 of the sheath 200 may be positioned on the sheath body 210 in any suitable manner. For example, the electrodes 281, 282, 283 may extend around the circumference of the sheath body 210 or may extend less than all the way around the circumference of the sheath body 210. For example, the electrodes 281, 282, 283 may radially extend around the sheath body 210 from about 10 degrees to about 360 degrees, such as from about 10 degrees to about 20 degrees, from about 150 degrees to about 210 degrees, or about 180 degrees. Each electrode 281, 282, 283 may extend around the sheath body 210 the same or a different amount. Preferably, each electrode 281, 282, 283 extends around the sheath body 210 the same amount. Preferably, each electrode 281, 282, 283 extends radially around the sheath body 210 about 180 degrees, or about half-way around the circumference of the sheath body 210, to all the way around the circumference of the sheath body 210, or about 360 degrees.

The electrodes 281, 282, 283 may be made of any suitable material. Suitable materials for implantable electrodes are well-known to those of skill in the art. Materials suitable for deep brain stimulation electrodes are suitable materials for electrodes of the catheters described herein. In embodiments, the electrodes are made from platinum or a platinum iridium alloy.

The electrodes 281, 282, 283 may have any suitable thickness. For example, the electrodes 281, 282, 283 may have a thickness from about 100 microns to about 3 millimeters, such as from about 200 microns to about 2 millimeters. The electrodes 281, 282, 283 may be formed from a foil.

Each electrode 281, 282, 283 may be discretely electrically coupled to one or more electrical contacts. Each electrode may be electrically coupled to a discrete contact by an electrical conductor, such as a wire. The conductors may extend within the sheath body 210 and may branch off from the sheath body 210 in proximity to the proximal end of the sheath body 210. The conductors may comprise an electrically insulating coating or outer surface. Conductors that branch off the sheath body 210 may be braided or twisted to form a cable 295. The cable may comprise an electrically insulating outer material.

In embodiments, an assembly comprises the sheath 200, and a catheter. In embodiments, an assembly comprises the sheath 200, the burr hole device 100, and a catheter. In embodiments, an assembly comprises the sheath 200, the burr hole device 100, a catheter, and a cranial port device. In embodiments, an assembly comprises the sheath 200, a catheter, and a cranial port device. The catheter may be a therapeutic fluid delivery catheter, a catheter of a ventriculoperitoneal (VP) shunt, a catheter of an external ventricular drain (EVD), a catheter for aspirating CSF, or the like. The assembly may comprise a VP shunt, an EVD, an infusion device, which may be an implantable infusion device, configured to connect to the therapeutic fluid delivery catheter, or the like.

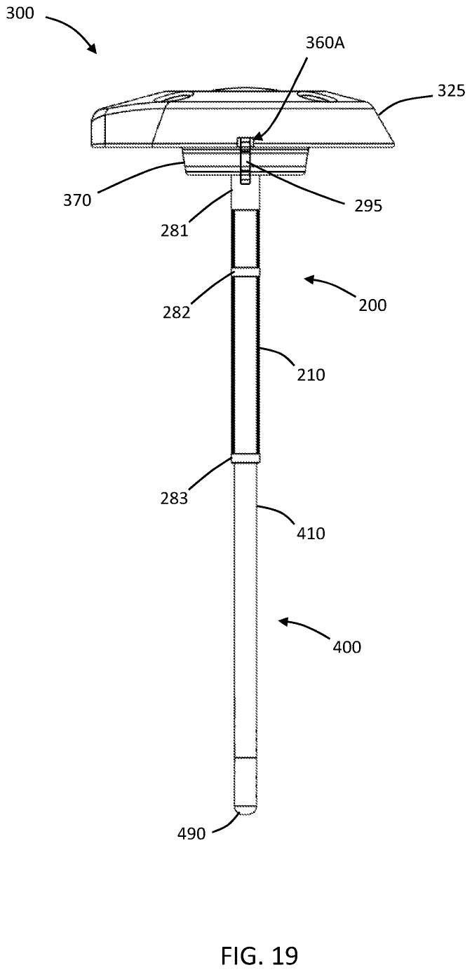

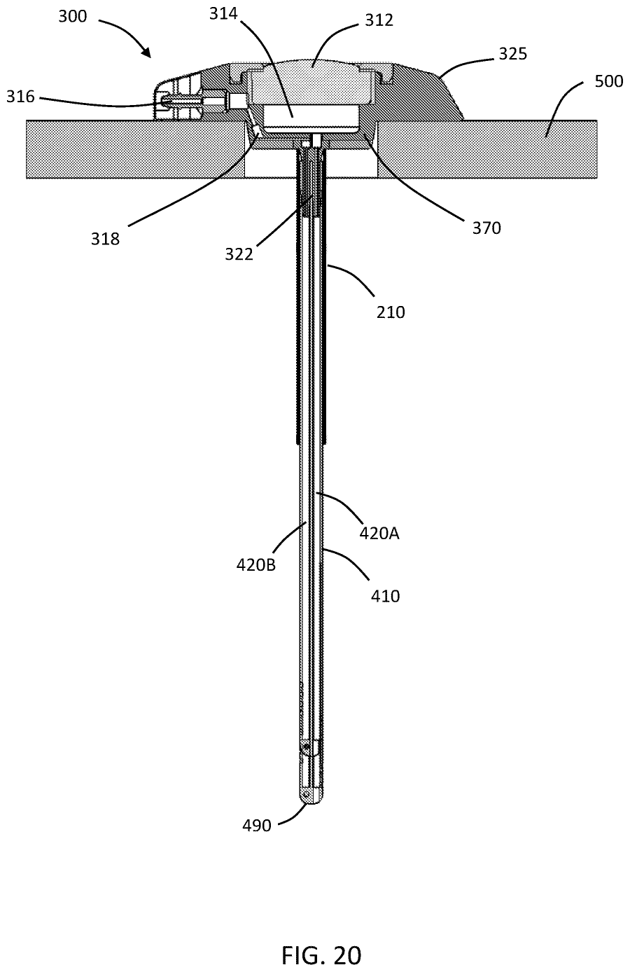

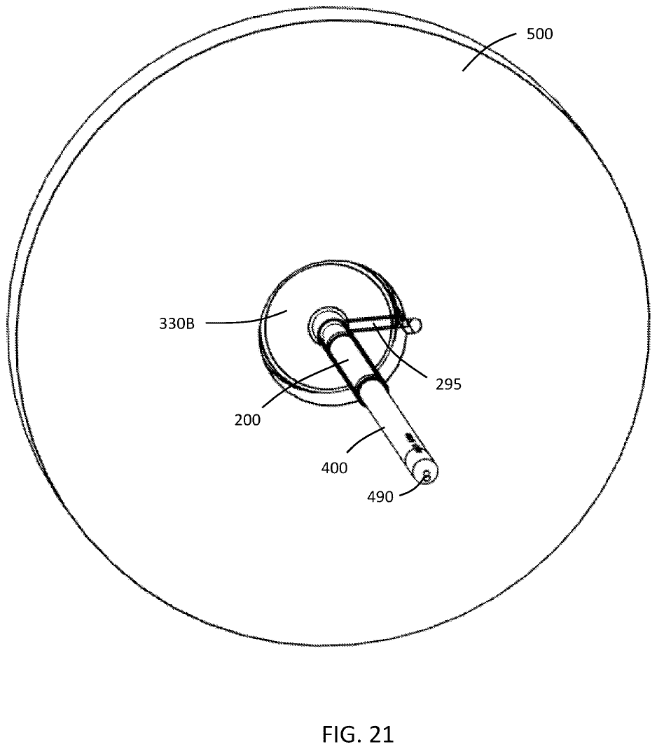

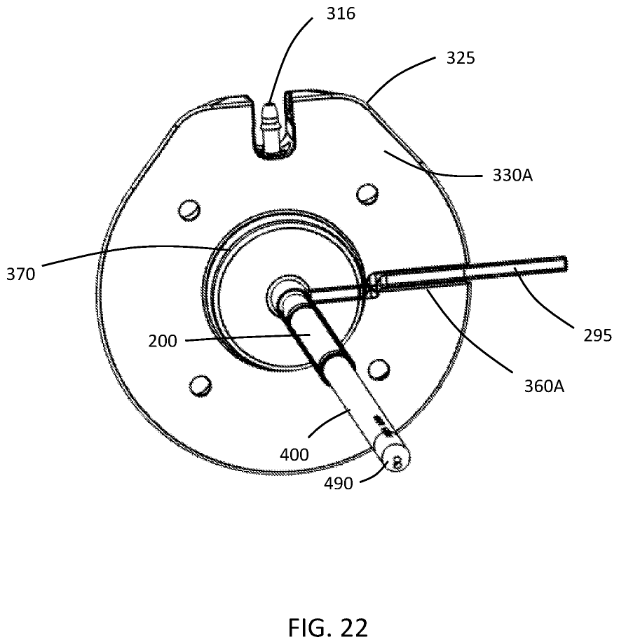

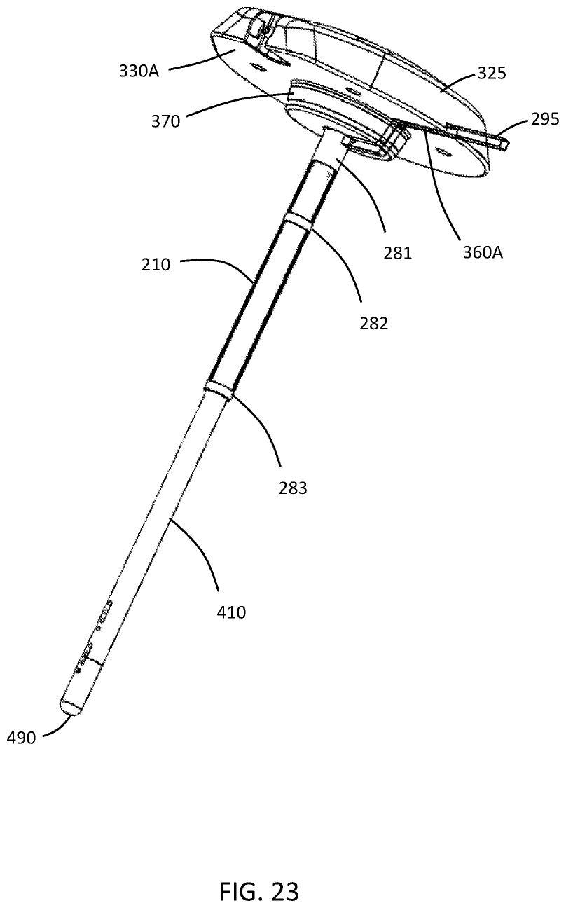

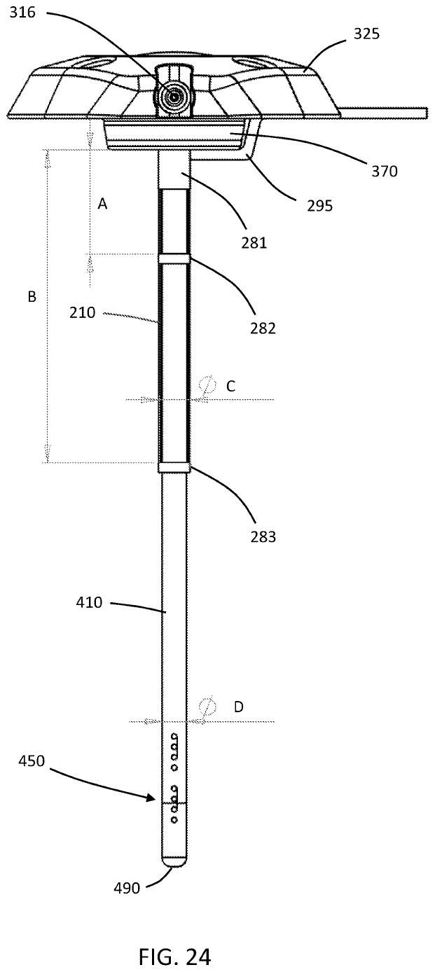

Referring now to FIGS. 18-26, assemblies including a sheath 200, a cranial port device 300, and a catheter 400 are shown. The assembly of FIGS. 18-24 includes the sheath 200 of FIG. 17. The assemblies of FIGS. 18-26 include a cranial port device 300 having two fluid pathways, and a dual lumen catheter 400. However, the assemblies described herein may comprise any suitable cranial port device having any suitable number of fluid pathways, such as one or two pathways, and any suitable catheter having any suitable number of lumens, such as one or two.

The cranial port device 300 depicted in the assemblies of FIGS. 18-26 may be a cranial port device as described in U.S. 2022/016338-A1, entitled IMPLANTABLE CRANIAL MEDICAL DEVICE, filed on Jul. 15, 2021, and published on Jan. 20, 2022, or a modified version thereof.

The depicted cranial port device 300 comprises a body defining an upper flange portion 325 and a lower portion 370. The lower portion 370 is configured to be received by a burr hole in the skull 500 of the subject. The upper flange portion 325 is configured to rest on the skull 500 adjacent to the burr hole. Preferably, the lower portion 370 of the cranial port device 300 has a clearance of 1 millimeter or less when disposed in the burr hole to help stabilize the device 300 when implanted. In embodiments, the lower portion 370 of the cranial port device 300 has an outer diametric dimension in a range from 10 millimeters to 20 millimeters, which outer diametric dimension may depend on the size of the burr hole. In embodiments, the lower portion 370 of the cranial port device 300 has an outer diametric dimension in a range from 10 millimeters to 20 millimeters. In embodiments, the lower portion 370 of the cranial port device 300 has an outer diametric dimension in a range from 10 millimeters to 14 millimeters, such as from about 11 millimeters to about 13 millimeters. Typically, the outer surface of the lower portion 370 is generally cylindrical as depicted in FIGS. 18-26 or frustoconical.

When the cranial port device 300 is implanted, a bottom surface 330B of the lower portion 370 of the device 300 preferably does not extend substantially beyond the bottom of skull when received in the burr hole. More preferably, the bottom surface 330B of the lower portion 370 of the device 300 preferably does not extend substantially beyond the bottom of skull. Accordingly, the height of lower portion 370 may vary depending on the thickness of the skull of the subject into which the cranial port device 300 is implanted. As an example, a thickness of a human adult skull may typically be in a range from about 6.5 millimeters to about 7 millimeters.

In some embodiments, the height of the lower portion 370 of the cranial port device 300 (distance from the bottom surface 330A of the upper flange portion 325 to the bottom surface 330B of the lower portion 370) is in a range from about 3 millimeters to about 7 millimeters. For example, the height of the lower portion 370 may be in a range from about 4 millimeters to about 6 millimeters or from about 4.5 millimeters to about 5.5 millimeters.

The upper flange portion 325 is configured to be disposed on the surface of the skull adjacent to the burr hole. That is, the bottom surface 330A of the upper flange portion 325 may rest on the skull when the device 300 is implanted. The upper flange portion 325 has an outer diametric dimension that is greater than the diameter of the burr hole. In embodiments, the outer diametric dimension of the upper flange portion 370 is in a range from 15 millimeters to 30 millimeters.

The bottom surface 330A of the upper flange portion 325 defines a bottom groove 360A configured to receive a cable 295 of the sheath 200. The bottom groove 360A extends from a lateral edge 121 of the bottom surface 330A towards the bottom surface 330A abutting the lower portion 370. The cable 295 may be protected by the bottom groove 360A when implanted, rather than being pinched between the upper surface of the skull and the bottom surface 330A of the upper flange portion 325 of the cranial port device 300.

While not shown, the lower portion 370 of the device 300 may define a side groove that may run the length of the lower portion 370. The side grove may intersect with and meet the bottom groove 160A. The side groove may be configured to receive the cable 295 such that the cable. The cable may be positioned in the side groove between the side of the burr hole and the body of the lower portion 370 when the cranial port device 300 is implanted. When clearances are small between the lower portion 370 and the burr hole, the cable 295 may be protected in the side groove, as opposed to being pinched between the lower portion 370 of the device 300 and the side of the burr hole. The cranial port device 300 may optionally comprise bottom groove on the bottom surface 330B of the lower portion 370 of the cranial port device 300. The bottom groove may intersect and meet with side groove.

The cranial port device 300 includes two fluid flow pathways. The first pathway runs from a opening in the upper flange portion 325 (shown with self-sealing septum 312 disposed across the opening) to a first lumen in a brain catheter connector 322 extending from the bottom of the lower portion 370. The first pathway may be used to withdraw fluid from the brain of the subject, or to inject fluid into the brain of the subject, through the catheter 200. The second fluid pathway extends from a lumen of an infusion catheter connector 316 to a second lumen in the brain catheter connector 322. The second pathway may be used to infuse fluid into the brain of the subject when connected to the catheter 200. An infusion catheter may be coupled to the infusion catheter connector 316 on one end and may be coupled to an infusion device, such an implantable infusion device, on the other end.

When coupled to the brain catheter connector 322, a first lumen 420A of the catheter 400 is fluidly coupled to the first fluid pathway of the cranial port device 300 and a second lumen 420A of the catheter 400 is fluidly coupled to the second fluid pathway of the cranial port device 300. Openings 450 are defined in the catheter body 410. Each opening 450 is in fluid communication with either the first 420A or second 420B lumens of the catheter 400 to allow fluid to flow into the brain from the catheter 400 or to flow into the catheter 400 from the brain when the catheter 400 is implanted and coupled to the cranial port device 300.

In embodiments, the distal end 490 of the catheter 400 is positioned in a CSF-containing space of the brain when implanted. Preferably, the distal end 490 of the catheter 400 is positioned in a lateral ventricle.

The catheter 200 may be a catheter as described in U.S. 2022/016338-A1, entitled IMPLANTABLE CRANIAL MEDICAL DEVICE, filed on Jul. 15, 2021, and published on Jan. 20, 2022, or any other suitable catheter.

A proximal portion of the catheter 400 is received in the lumen defined by the sheath body 210. The sheath body 210 and catheter 400 are configured to be intracranially implanted beneath the skull 500 of a subject. When implanted, one or more electrodes 281, 282, 283 of the sheath 200 are preferably in contact with parenchymal tissue of the brain.

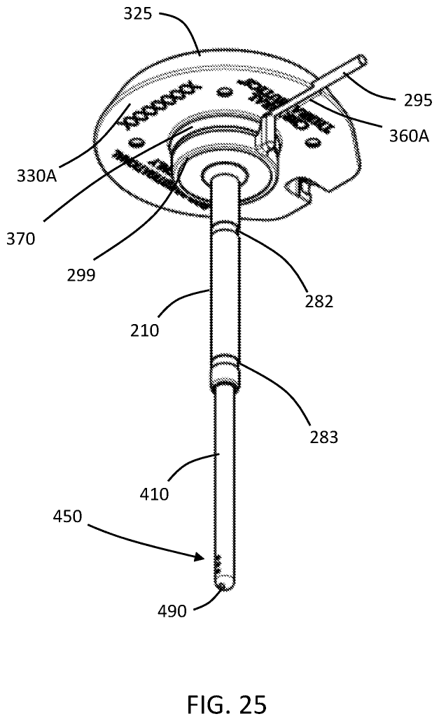

With reference to FIG. 24, the distance A from the bottom of the lower portion 370 of the cranial port device to the top of electrode 282 of the sheath in the assembly is preferably sufficient to cause the electrode 282 to be implanted in the brain when the assembly is implanted. In embodiments, the distance A is in a range from 5 millimeters to 15 millimeters, such as 8 millimeters to 12 millimeters, or about 10 millimeters.

The distance B from the bottom of the lower portion 370 of the cranial port device to the top of electrode 283 of the sheath in the assembly is preferably sufficient to cause the electrode 283 to be implanted in the brain when the assembly is implanted. In embodiments, the distance B is in a range from 25 millimeters to 35 millimeters, such as 28 millimeters to 32 millimeters, or about 30 millimeters.

The outer diameter C of the sheath is preferably small to minimize damage to the brain when implanted and is not substantially larger than the outer diameter D of the catheter. In embodiments, the outer diameter C of the sheath is in a range from 2 millimeters to 4 millimeters, such as 2.5 millimeters to 3.5 millimeters, or about 3 millimeters. In embodiments, the outer diameter D of the catheter is in a range from 1.75 millimeters to 3 millimeters, such as 2 millimeters to 2.5 millimeters, or about 2.5 millimeters or about 2.33 millimeters.

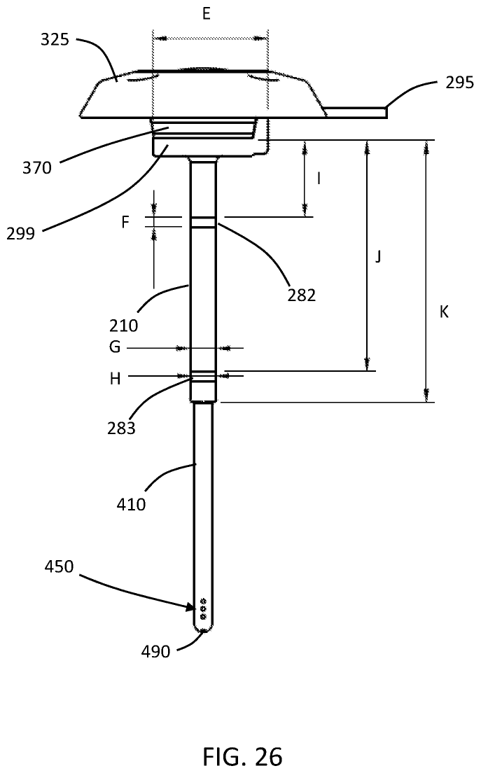

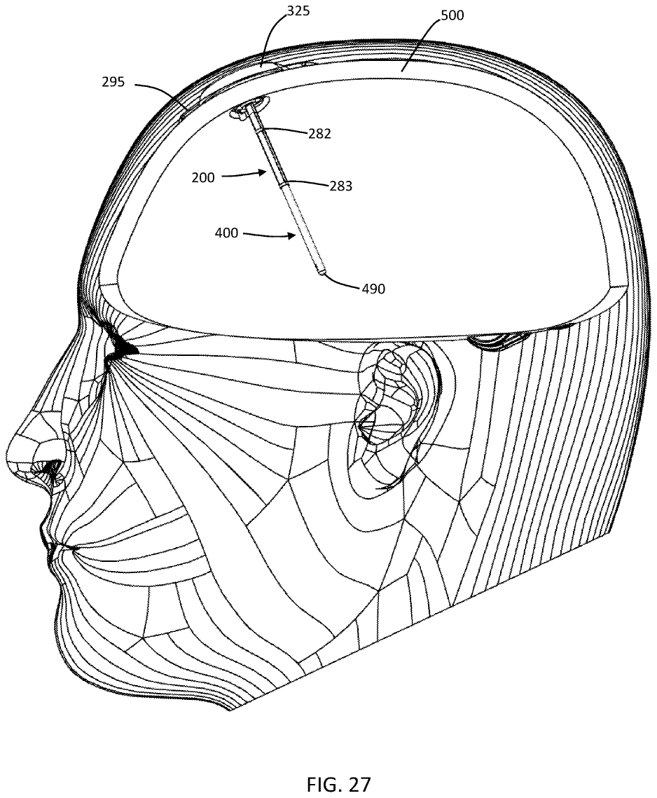

In FIGS. 25 and 26, the sheath 200 comprises an annular place 299 at the proximal end of the sheath body 210. The cable 295 extends away from the annular plate 299. The annular plate 299 abuts the bottom surface of the lower portion 370 of the cranial port device 300 when implanted.

With reference to FIG. 26, the width or diametric dimension E of the annular plate 299 and portion of cable 295 positioned below the lower portion 370 of the cranial port device in the assembly is preferably sufficiently small to fit within a burr hole when implanted. In embodiments, the width E is in a range from 14 millimeters to 15 millimeters, such as from 14.5 millimeters to 14.95 millimeters, or about 14.9 millimeters or about 14.89 millimeters.

The electrodes 282, 283 may have any suitable height. In embodiments, the height F of electrode 282 is in a range from 0.5 millimeters to 2 millimeters, such as from 0.8 millimeters to 1.6 millimeters, or about 1.2 millimeters.

The outer diameter G of the sheath is preferably small to minimize damage to the brain when implanted and is not substantially larger than the outer diameter of the catheter body 410. In embodiments, the outer diameter G of the sheath is in a range from 2 millimeters to 4 millimeters, such as 2.5 millimeters to 3.8 millimeters, or about 3.4 millimeters.

In embodiments, the outer diameter F of the electrode 283 disposed on the sheath body 210 is less than the outer diameter of the sheath body 210. In embodiments, the outer diameter F of the electrode 283 is in a range from 2 millimeters to 4 millimeters, such as 2.5 millimeters to 3.5 millimeters, or about 3 millimeters or about 3.2 millimeters.

The distance I from the bottom of the lower portion 370 of the cranial port device to the top of electrode 282 of the sheath in the assembly is preferably sufficient to cause the electrode 282 to be implanted in the brain when the assembly is implanted. In embodiments, the distance A is in a range from 5 millimeters to 15 millimeters, such as 8 millimeters to 12 millimeters, or about 10 millimeters.

The distance J from the bottom of the lower portion 370 of the cranial port device to the top of electrode 283 of the sheath in the assembly is preferably sufficient to cause the electrode 283 to be implanted in the brain when the assembly is implanted. In embodiments, the distance B is in a range from 25 millimeters to 35 millimeters, such as 28 millimeters to 32 millimeters, or about 30 millimeters.

The distance K from the bottom of the lower portion 370 of the cranial port device to the distal end of the sheath body 210 in the assembly is shorter than the length of the catheter. In embodiments, the distance K is in a range from 30 millimeters to 40 millimeters, such as 32 millimeters to 36 millimeters, or about 34 millimeters.

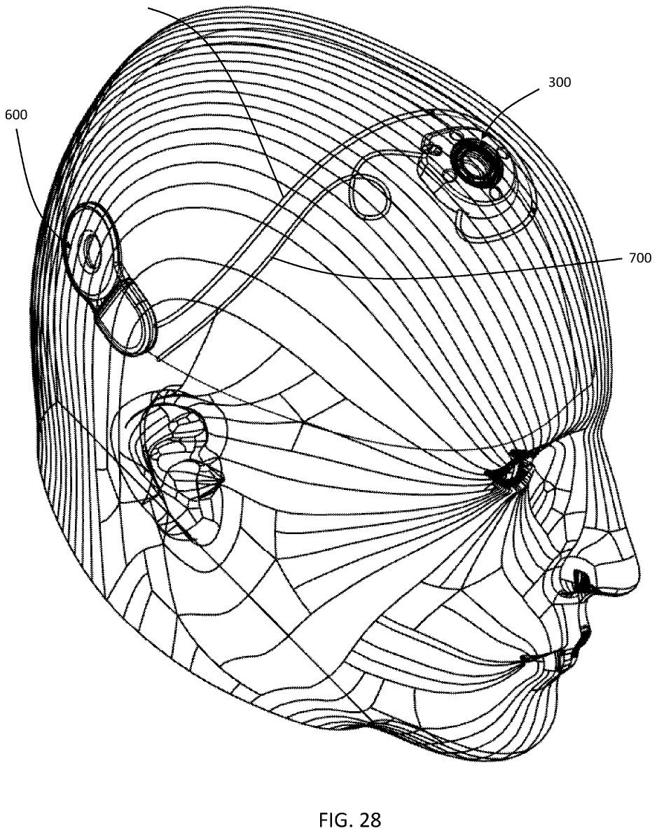

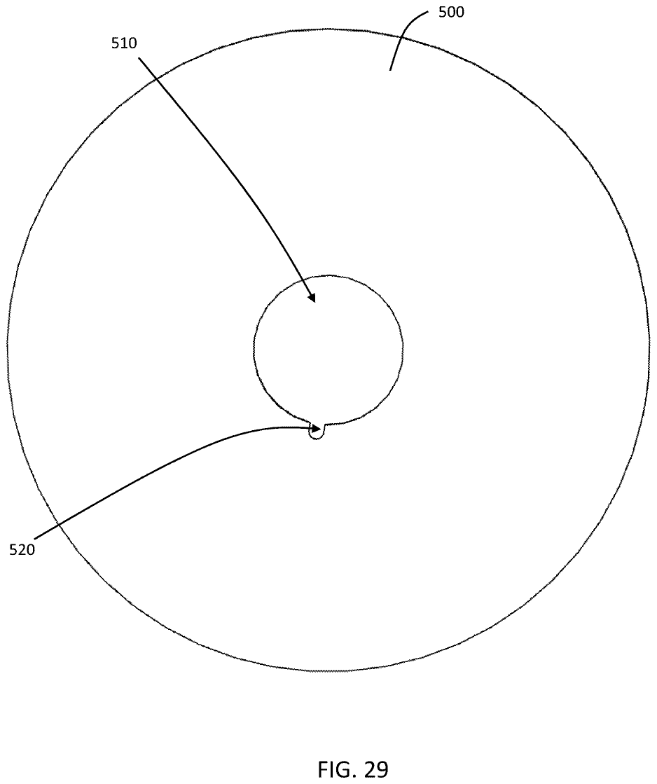

Referring now to FIGS. 27 and 28, a system comprising a cranial port device 300, a sheath 200, a catheter 400, and signal apparatus 600 are shown implanted in a subject. Components of the system shown in FIGS. 27 and 28 may be similar to the assemblies described regarding FIGS. 18-26. The systems shown in FIGS. 27 and 28 may have some of or all the features or elements of the assemblies FIGS. 18-26. While not shown, the system may also comprise a burr hole device as described herein, or a burr hole device may be substituted for the cranial port device.

As illustrated, the catheter 400 is received within the sheath 200 and operably coupled to the cranial port device 300. Electrodes 282, 283 and the distal end 490 the catheter 400 are positioned in the brain of the subject. The upper flange portion 325 of the cranial port device 300 is positioned on the skull 500 of the subject. The cable 295 of the sheath 200 exits a bottom groove of the upper flange portion 325, extends along the surface of the skull 500 and is operatively coupled to signal apparatus 600. The signal apparatus 600 is shown implanted between the scalp and the skull 500 behind the ear of the subject but may be implanted in any other suitable location. The signal apparatus 600 may be configured to one or more of store, process, and transmit brain activity data received by electrodes 282, 283, etc. Signal apparatus 600 may be configured to transmit data to a device outside of the patient.

An infusion catheter 700 is coupled to the infusion catheter port of the cranial port device 300 and extends across the surface of the skull and subcutaneously to an implantable infusion device (not shown).

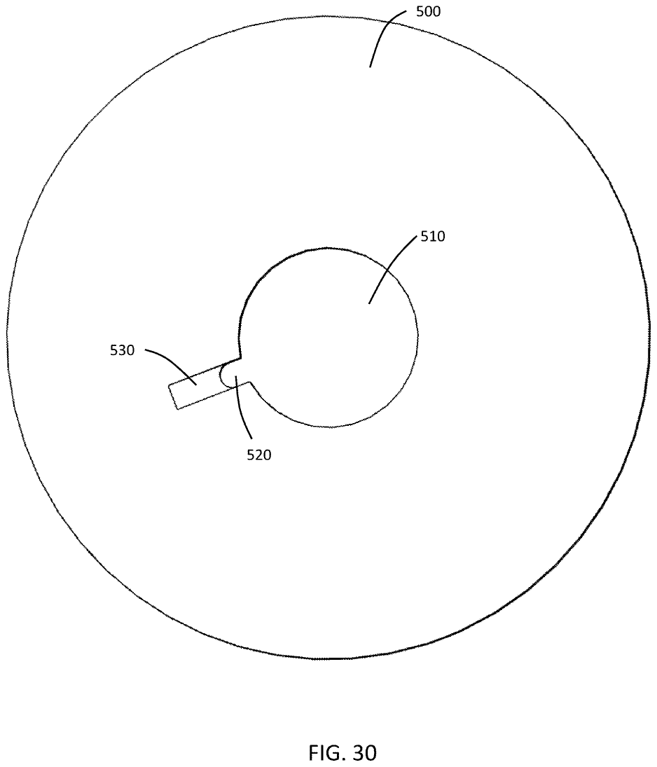

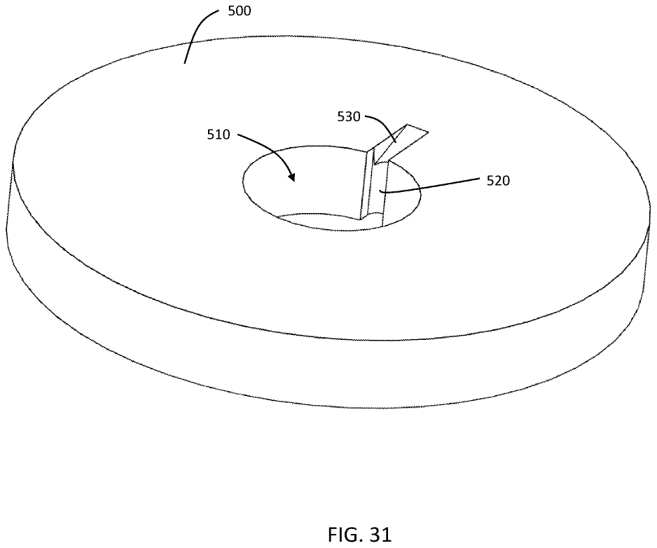

Referring now to FIGS. 29-31 burr holes 510, 520 in a portion of a skull 500 are shown to illustrate a method that may be employed to implant the burr hole device, catheter and sheath, cranial access port, and assemblies described herein.

A first burr hole 510 is created in the skull 500. The edge of the skull 500 defines the first burr hole 510. The first burr hole 510 has a diameter sufficiently large to accommodate the device to be implanted in the burr hole 510. For example, the diameter of the burr hole 510 may be suitable for accommodating passage of a catheter and sheath body, accommodating a lower portion of a burr hole device or a lower portion of a cranial port device, or the like. In embodiments, the first burr hole 510 has a diameter in a range from about 4 millimeters to about 20 millimeters, such as from about 5 millimeters to about 15 millimeters. In embodiments, the first burr hole 510 has a diameter of about 5 millimeters. In embodiments, the first burr hole 510 has a diameter of about 15 millimeters.

A second burr hole 520 is created through the skull 500. The second burr hole 520 has a smaller diameter than the first burr hole 510. The second burr hole 520 is configured to receive a cable, such as a cable of a sheath as described herein. By feeding the cable through the second burr hole 520 the cable may avoid being pinched by any device, such as a cranial port device or a burr hole device, inserted into the first burr hole 510. In embodiments, the diameter of the second burr hole is in a range from 1 millimeter to 10 millimeters.

The first 510 and second 520 burr holes partially overlap. The second burr hole 520 forms a groove along the edge of the first burr hole 520 along the thickness of the skull 500. Such a configuration provides access to the second burr hole 520 through the first burr hole 510.

In embodiments, the first burr hole 510 is formed through the skull 500 prior to the second burr hole 520 being formed. In embodiments, the first burr hole 510 is formed through the skull 500 after to the second burr hole 520 being formed.

A trench 530 may be created in the top surface of the skull 500. The trench 530 has a first end and a second end. The first end of the trench 530 intersects the second burr 520, and the trench 530 extends away from the second burr hole 520 towards the second end. The trench 530 has a first depth at the first end and has a second depth at the second end. The first depth is greater than the second depth.

The trench 530 provides a ramp to allow the cable to exit the second burr hole 520 at an angle less than 90 degrees, which may prevent damage to conductors running through the cable.

The figures and description above describe burr hole devices, catheter access ports, catheters, sheaths, signal apparatus, and assemblies and systems comprising one or more of the burr hole devices, catheter access ports, catheters, sheaths, and signal apparatus, as well as methods of use and methods of implantation of such devices, assemblies, and systems. The devices, assemblies, systems, and methods described herein, among other things, allow or facilitate implanting a catheter into a brain of a subject and implanting a recording electrode into the brain of the subject. The catheter may be used to provide treatment to the subject, through delivery of therapeutic fluids to the brain, through removal of fluid from the brain, or through delivery of fluid to the brain and removal of fluid from the brain. The recording electrode may be used to monitor brain activity signals, which may or may not be related to the disease being treated. When related to the disease being treated, the monitored brain activity data may be used to adjust treatment (e.g., delivery of therapeutic fluid or removal of cerebral spinal fluid) based on the data.

The electrodes of the sheath, assembly, or systems described herein may be configured to detect electroencephalogram (EEG) signals. The sheath, assembly, or systems may comprise one or more electrodes. One or more electrodes may be a recording electrode. One or more electrodes may be a ground or reference electrode.

The sheath, assembly, or systems may comprise any suitable number of electrodes, such from 1 electrode to about 64 electrodes. For example, 2 to 32 electrodes, such 2 to 16 electrodes, or 2 to 10 electrodes.

In embodiments, most or all the electrodes are positioned such that they will be placed in white or grey matter of the brain when implanted. However, it is not necessary that all the electrodes be placed in the white or grey matter. If an electrode is not placed in white or grey matter, the recording from that electrode may continue to be captured and ultimately ignored or captured and considered. Alternatively, recording from that electrode may be inactivated. In embodiments, one or more electrodes are placed on a surface of the brain or above the brain as a ground or reference electrode.

Preferably, at least two electrodes are configured to be placed in white or grey matter. When multiple electrodes record signals from white or grey matter, coherent changes in activity between electrodes may be a powerful way to track more global changes. In some embodiments, the excitable state of a neural network is determined by monitoring a small neuronal population. The more excitable the small neuronal population, the higher the probability for activity to propagate throughout the network causing an ‘avalanche’ of activity. Such monitoring may be valuable for general brain state monitoring and may be particularly valuable for monitoring a brain state to predict a seizure.

In embodiments, the excitable state of a neural network is determined by monitoring brain activity at multiple brain locations (termed nodes). Interaction in the local field activity between the nodes can be used to determine the brain excitability. Such monitoring may be valuable for general brain state monitoring and may be particularly valuable for monitoring a change in brain state to predict a seizure.

The electrodes and associated signal processing apparatus may be configured in any suitable manner. For example, the electrodes and associated signal apparatus may be configured in differential mode or referential mode.

In differential mode, the system comprises an active electrode, a reference electrode, and a ground. The signal difference between an active electrode and a reference electrode may be amplified. The reference electrode may be a common reference for more than one active electrode. The reference electrode is preferably positioned a substantial distance from an active electrode and from the ground. In differential mode, the system may be configured to detect small differences between electrode pairs and may be less likely to be affected by large artifacts originating near the ground electrode. However, the system may not be particularly effective at detecting larger common signals.

Preferably, the system is configured to detect larger common signals. Larger common signals may be associated with an overall brain state or with a seizure.

To detect larger common signals, the system may be configured in referential mode, which may also be referred to as single-ended mode. Referential mode may use a single active electrode per amplifier. There may be more than one active electrode. In referential mode, the output of the active electrode is amplified relative to the ground electrode, as opposed to the reference electrode in differential mode. The ground is preferably placed a substantial distance from the active electrode, which may result in amplification of signals that affect larger parts of the brain. While being effective at detecting larger common signals, referential mode may be sensitive to artifacts. Proper placement of the ground electrode may mitigate some issues associated with artifacts.

The signal apparatus may comprise a power source, such as a battery, which may be rechargeable, or may be wirelessly powered. If the signal apparatus is wirelessly powered, the signal apparatus preferably includes an inductive coil, solenoid, or other suitable components to be wirelessly powered by an external apparatus and to transmit data regarding the signals recorded by the electrodes to the external apparatus.

In embodiments, the signal apparatus is implanted at a location where it may inductively couple with a device external to the subject. For example, the signal apparatus may be positioned under the scalp of the subject near an ear of the subject. Such positioning may allow the external device to be comfortably worn on or around the ear of the subject to provide suitable inductive coupling to power the signal apparatus and to wirelessly transmit data regarding the signals recorded by the electrodes from the signal apparatus to the external device. The external device may then transfer the data directly to the cloud or via another device, such as a smart phone, a personal computer, or the like, which may then transfer the data to a server in the cloud, or the like.

The signal apparatus may be configured to continuously transmit EEG data derived from an intracranial electrode. The signal apparatus may be configured to continuously transmit the EEG data for a long duration of time. The signal apparatus may be configured to transmit relatively unfiltered data containing a broad amount of relevant brain signal. That is, the signal apparatus may transmit data regarding a majority of the captured EEG data. For example, the EEG data corresponding to the transmitted data may not have been bandpass filtered. As another example, the subsets of the EEG data are not extracted for transmission. Rather, the majority of the EEG data is transmitted by the signal apparatus. The signal apparatus may be configured to continuously transmit data for 1 day or more, 1 week or more, 1 month or more, or 1 year or more.

The external device may be configured to continuously receive EEG data derived from an intracranial electrode. The external device may be configured to continuously receive the EEG data for a long duration of time. The external device may be configured to continuously receive data for 1 day or more, 1 week or more, 1 month or more, or 1 year or more.

The EEG data may be used for any suitable purpose. The EEG data may be used to treat, monitor, or treat and monitor a disease for which the catheter may be employed. The EEG data may be used to identify, classify, or predict a brain state associated with the disease being treated or that has been treated. The EEG data may be used to identify, classify, or predict a brain state that is not associated with the disease being treated or that has been treated. The EEG data may be used to identify, classify, or predict a brain state associated with the disease being treated or that has been treated and a brain state that is not associated with the disease being treated or that has been treated. The EEG data may be used to identify, classify, or predict a psychological brain state associated with the disease being treated or that has been treated and a brain state that is not associated with the disease being treated or that has been treated.

The EEG data may be used to develop or train an AI model that may identify, classify, or predict a brain state. The EEG data may be input into an AI model that may identify, classify, or predict a brain state. The brain state may or may not be associated with the disease being treated or that has been treated. The brain state may be a psychological brain state.

Development, training, refining, and utilizing AI models based on EEG data to identify, classify, or predict a brain state, such as a psychological brain state are discussed in U.S. patent application Ser. No. 17/380,694, entitled MONITORING BASED ON CONTINUOUS INTRACRANIAL EEG ACTIVITY, filed on Jul. 20, 2021, and naming Cerebral Therapeutics, Inc. as an Applicant, and U.S. Provisional Patent Application No. 63/280,367, entitled DEVELOPMENT AND IMPLEMENTATION OF PSYCHOLOGICAL STATE MODEL, filed on Nov. 17, 2021, and naming Cerebral Therapeutics, Inc. as an Applicant, which provisional applications are hereby incorporated herein by reference in their entireties to the extent that they do not conflict with the disclosure presented herein.

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

Example 1: A burr hole device comprising a body having a top surface and a bottom surface and defining a lumen extending from the top surface to the bottom surface, wherein the lumen is configured to receive a catheter configured to allow fluid to flow to or from a brain of a subject, wherein at least a portion of the bottom surface is configured to be disposed on a surface of a skull adjacent to a burr hole in the skull of the subject, wherein the body further defines a top groove along the top surface, wherein the top groove extends from the lumen towards a lateral edge of the top surface of the body, wherein the top groove is configured to receive the catheter, wherein the body further defines a bottom groove along the bottom surface, wherein the bottom groove extends from a lateral edge of the bottom surface towards the lumen, wherein the bottom groove is configured to receive a cable.

Example 2: The burr hole device of Example 1, wherein the body defines an upper flange portion and a lower portion, wherein the lower portion is configured to be placed within the burr hole and the upper flange portion is configured to be disposed on the surface of the skull.

Example 3: The burr hole device of Example 2, wherein the bottom groove is disposed on a bottom surface of the upper flange portion.

Example 4: The burr hole device of Example 3, wherein the lower portion of the body defines a side groove along a side of the lower portion, wherein the side groove meets with the bottom groove.

Example 5: The burr hole device of any one of Examples 2 to 5, wherein the lower portion of the body is configured to have a clearance of 1 millimeter or less when disposed in the burr hole.

Example 6: The burr hole device of any one of Examples 2 to 5, wherein the lower portion of the body has an outer diametric dimension from 10 millimeters to 20 millimeters.

Example 7: The burr hole device of any one of Examples 2 to 6, wherein the upper flange portion of the body has an outer diametric dimension from 15 millimeters to 30 millimeters.

Example 8: A burr hole device comprising a body having a top surface and a bottom surface and defining a lumen extending from the top surface to the bottom surface, wherein the lumen is configured to receive a catheter configured to infuse fluid to, or withdraw fluid from, a brain of a subject, wherein at least a portion of the bottom surface is configured to be disposed on a surface of a skull adjacent to a burr hole in the skull of the subject, wherein the body further defines a first top groove along the top surface, wherein the first top groove extends from the lumen towards a lateral edge of the top surface of the body, wherein the first top groove is configured to receive the catheter, wherein the body further defines a second top groove along the top surface, wherein the second top groove extends from the lumen towards a lateral edge of the top surface of the body, wherein the second top groove is configured to receive a cable

Example 9: The burr hole device of Example 8, wherein the body defines an upper flange portion and a lower portion, wherein the lower portion is configured to be placed within the burr hole and the upper flange portion is configured to be disposed on the surface of the skull.

Example 10: The burr hole device of Example 9, wherein the lower portion of the body is configured to have a clearance of 1 millimeter or less when disposed in the burr hole.

Example 11: The burr hole device of Example 9, wherein the lower portion of the body has an outer diametric dimension from 10 millimeters to 20 millimeters.

Example 12: The burr hole device of any one of Examples 9 to 11, wherein the upper flange portion of the body has an outer diametric dimension from 15 millimeters to 30 millimeters.

Example 13: A burr hole device comprising a body having a top surface and a bottom surface and defining a lumen extending from the top surface to the bottom surface, wherein the lumen is configured to receive a catheter configured to infuse fluid to, or withdraw fluid from, a brain of a subject, wherein at least a portion of the bottom surface is configured to be disposed on a surface of a skull adjacent to a burr hole in the skull of the subject, wherein the body further defines a top groove along the top surface, wherein the top groove extends from the lumen towards a lateral edge of the top surface of the body, wherein the top groove comprises a lower groove portion and an upper groove portion, the lower groove portion having a smaller diametric dimension than the upper groove portion, wherein the lower groove portion is configured to receive a cable, and wherein the upper groove portion is configured to receive the catheter such that the catheter is positioned over the cable when the both the cable and the catheter are received in the top groove.

Example 14: The burr hole device of Example 13, wherein the body defines an upper flange portion and a lower portion, wherein the lower portion is configured to be placed within the burr hole and the upper flange portion is configured to be disposed on the surface of the skull.

Example 15: The burr hole device of Example 14, wherein the lower portion of the body is configured to have a clearance of 1 millimeter or less when disposed in the burr hole.

Example 16: The burr hole device of Example 14, wherein the lower portion of the body has an outer diametric dimension from 10 millimeters to 20 millimeters.

Example 17: The burr hole device of any one of Examples 13 to 16, wherein the upper flange portion of the body has an outer diametric dimension from 15 millimeters to 30 millimeters.

Example 18: An assembly comprising: (i) the burr hole device of any one of Examples 1 to 17; the catheter; and a sheath comprising (i) a sheath body defining a sleeve lumen configured to receive the catheter, (ii) a recording electrode disposed on the sheath body, (iii) the cable, and (iv) a conductor electrically coupled to the recording electrode and extending in the cable.

Example 19: A method comprising: providing the assembly of Example 18; disposing the burr hole device in or about a burr hole of a subject; inserting the catheter through the lumen of the burr hole device; inserting the catheter through the sheath lumen; implanting a distal end of the catheter in a brain of the subject; implanting the recording electrode of the sheath beneath the skull of the subject; inserting the cable of the sheath in the bottom groove, the second top groove, or the bottom portion of the top groove; and inserting the catheter in the top groove, the first top groove, or the top portion of the top groove.

Example 20: The method of Example 19, wherein implanting the recording electrode of the sheath beneath the skull of the subject comprises implanting the electrode in the brain of the subject.

Example 21: The method of Example 19 or 20, wherein implanting a distal end of the catheter in the brain of the subject comprises implanting the distal end of the catheter in a cerebral ventricle.

Example 22: A burr hole device comprising (i) a body having a top surface and a bottom surface and defining a lumen extending from the top surface to the bottom surface, wherein the body defines an upper flange portion and a lower portion, wherein the lower portion is configured to be placed within the burr hole and the upper flange portion is configured to be disposed on the surface of the skull, wherein the lumen is configured to receive a catheter configured to infuse fluid to, or withdraw fluid from, a brain of a subject; and (ii) an electrode disposed on the bottom surface of the lower portion.

Example 23: The burr hole device of Example 22, wherein the bottom surface of the lower portion is configured to be placed on a surface of a brain or above the surface of the brain when the burr hole device is implanted.

Example 24: The burr hole device of Example 22, wherein the bottom surface of the lower portion extends from 3 millimeters to 7 millimeters from the bottom surface of the upper flange portion.

Example 25: The burr hole device of any one of Examples 22 to 24, wherein the lower portion of the body is configured to have a clearance of 1 millimeter or less when disposed in the burr hole.

Example 26: The burr hole device of Example 25, wherein the lower portion of the body has an outer diametric dimension from 10 millimeters to 20 millimeters.

Example 27: The burr hole device of any one of Examples 22 to 26, wherein the upper flange portion of the body has an outer diametric dimension from 15 millimeters to 30 millimeters.

Example 28: The burr hole device of any one of Examples 22 to 27, further comprising an electrical interconnect electrically coupled to the electrode.

Example 29: The burr hole device of Example 28, wherein the electrical interconnect is configured to electrically couple an implantable medical lead.

Example 30: The burr hole device of any one of Examples 22 to 29, wherein the body further defines a top groove along the top surface, wherein the top groove extends from the lumen towards a lateral edge of the top surface of the body, wherein the top groove is configured to receive the catheter.

Example 31: The burr hole device of any one of Examples 22 to 30, wherein the top grove is configured to grippingly engage the catheter.

Example 32: The burr hole device of Example 31, wherein the top groove comprises a lower groove portion and an upper groove portion, the lower groove portion having a smaller diametric dimension than the upper groove portion, wherein the lower groove portion is configured to receive a cable, and wherein the upper groove portion is configured to receive the catheter such that the catheter is positioned over the cable when the both the cable and the catheter are received in the top groove.

Example 33: The burr hole device of Example 31 or 32, wherein the body further defines a second top groove along the top surface, wherein the second top groove extends from the lumen towards a lateral edge of the top surface of the body, wherein the second top groove is configured to receive a cable.

Example 34: The burr hole device of any one of Examples 22 to 33, wherein the body further defines a bottom groove along the bottom surface, wherein the bottom groove extends from a lateral edge of the bottom surface towards the lumen, wherein the bottom groove is configured to receive a cable.

Example 35: An assembly comprising: the burr hole device of any one of Examples 22 to 34; and the catheter.

Example 36: The assembly of Example 35, further comprising a sheath comprising (i) a sheath body defining a sleeve lumen configured to receive the catheter, (ii) a recording electrode disposed on the sheath body, (iii) the cable, and (iv) a conductor electrically coupled to the recording electrode and extending in the cable.

Example 37: A method comprising operatively coupling to signal apparatus the electrode disposed on the bottom surface of the lower portion of the body of the burr hole device according to any one of Examples 22 to 34.

Example 38: The method of Example 37, wherein the signal apparatus is implantable signal apparatus.

Example 39: A method of Example 37 or 38, further comprising: providing the assembly of Examples 35 or 36; disposing the burr hole device in or about a burr hole of a subject; inserting the catheter through the lumen of the burr hole device; and implanting a distal end of the catheter in a brain of the subject.

Example 40: The method of Example 39, further comprising inserting the catheter in the top groove, the first top groove, or the top portion of the top groove.

Example 41: The method of Example 39 or 40 comprising: providing the assembly of Example 36; inserting the catheter through the sheath lumen; implanting the recording electrode of the sheath beneath the skull of the subject; and inserting the cable of the sheath in the bottom groove, the second top groove, or the bottom portion of the top groove.

Example 42: The method of Example 41, wherein implanting the recording electrode of the sheath beneath the skull of the subject comprises implanting the electrode in the brain of the subject.

Example 43: The method of any one of Examples 39 to 42, wherein implanting a distal end of the catheter in the brain of the subject comprises implanting the distal end of the catheter in a cerebral ventricle.

Example 44: A method comprising: providing the assembly of Example 35 or 36; disposing the burr hole device in or about a burr hole of a subject; inserting the catheter through the lumen of the burr hole device; and implanting a distal end of the catheter in a brain of the subject.

Example 45: The method of Example 44, further comprising inserting the catheter in the top groove, the first top groove, or the top portion of the top groove.

Example 46: The method of Example 44 or 45 comprising: providing the assembly of Example 36; inserting the catheter through the sheath lumen; implanting the recording electrode of the sheath beneath the skull of the subject; and inserting the cable of the sheath in the bottom groove, the second top groove, or the bottom portion of the top groove.

Example 47: The method of Example 46, wherein implanting the recording electrode of the sheath beneath the skull of the subject comprises implanting the electrode in the brain of the subject.

Example 48: The method of any one of Examples 44 to 47, wherein implanting a distal end of the catheter in the brain of the subject comprises implanting the distal end of the catheter in a cerebral ventricle.

Example 49: A burr hole device comprising: (i) a body defining an upper flange portion and a lower portion, wherein the lower portion is configured to be placed within the burr hole and the upper flange portion is configured to be disposed on the surface of the skull, wherein the body defines a cavity for receiving a cranial port device, wherein the lower portion defines an opening in communication with the cavity, wherein the opening is configured such that a catheter or a catheter connector of the port may be inserted therethrough; and an electrode disposed on the bottom surface of the lower portion.

Example 50: The burr hole device of Example 49, wherein a bottom surface of the lower portion is configured to be placed on a surface of a brain or above the surface of the brain when the burr hole device is implanted.

Example 51: The burr hole device of Example 49, wherein a bottom surface of the lower portion extends from 3 millimeters to 7 millimeters from a bottom surface of the upper flange portion.

Example 52: The burr hole device of any one of Examples 49 to 51, wherein the lower portion of the body is configured to have a clearance of 1 millimeter or less when disposed in the burr hole.

Example 53: The burr hole device of Example 52, wherein the lower portion of the body has an outer diametric dimension from 10 millimeters to 20 millimeters.

Example 54: The burr hole device of any one of Examples 49 to 53, wherein the upper flange portion of the body has an outer diametric dimension from 15 millimeters to 30 millimeters.

Example 55: The burr hole device of any one of Examples 49 to 54, further comprising an electrical interconnect electrically coupled to the electrode.

Example 56: The burr hole device of Example 55, wherein the electrical interconnect is configured to electrically couple an implantable medical lead.

Example 57: The burr hole device of any one of Examples 49 to 56, wherein the upper flange portion further defines a top groove, wherein the top groove extends from the cavity towards a lateral edge of the top surface, wherein the top groove is configured to receive the catheter.

Example 58: The burr hole device of Example 57, wherein the top groove comprises a lower groove portion and an upper groove portion, the lower groove portion having a smaller diametric dimension than the upper groove portion, wherein the lower groove portion is configured to receive a cable, and wherein the upper groove portion is configured to receive the catheter such that the catheter is positioned over the cable when the both the cable and the catheter are received in the top groove.

Example 60: The burr hole device of Example 58 or 59, wherein the upper flange portion further defines a second top groove along the top surface, wherein the second top groove extends from the cavity towards a lateral edge of the top surface of the body, wherein the second top groove is configured to receive a cable.

Example 61: The burr hole device of any one of Examples 49 to 60 and Examples 99 to 100, wherein the upper flange portion further defines a bottom groove along a bottom surface of the upper flange portion, wherein the bottom groove extends from a lateral edge of the bottom surface towards the cavity, wherein the bottom groove is configured to receive a cable.

Example 62: An assembly comprising: the burr hole device of any one of Examples 49 to 61 and Examples 99 to 100; and the cranial port device.

Example 63: The assembly of Example 62, wherein the cranial port device comprises an upper flange portion configured to rest on the upper flange portion of the burr hole device, and wherein the cranial port device comprises a lower portion configured to be received in the cavity of the burr hole device.

Example 64: The assembly of Example 63, wherein the cranial port device further comprises: a first fluid path; and a second fluid path, wherein the first fluid flow path extends from a first opening in the upper flange portion of the cranial port device to a first opening in the lower portion of the cranial port device, and wherein the second fluid flow path extends from a second opening in the upper flange portion of the cranial port device to a second opening in the lower portion of the cranial port device.

Example 65: The assembly of Example 62 to 64, further comprising a catheter operatively couplable or coupled to the cranial port device.

Example 66: The assembly of Example 65, wherein the catheter comprises a first lumen and a second lumen.

Example 67: The assembly of Example 66, wherein the first lumen of the catheter is in communication with the first fluid path of the cranial port device when the catheter is connected to the cranial port device, and wherein the second lumen of the catheter is in communication with the second fluid path of the cranial port device when the catheter is connected to the cranial port device.

Example 68: The assembly of any one of Examples 65 to 67, further comprising a sheath comprising (i) a sheath body defining a sleeve lumen configured to receive the catheter, (ii) a recording electrode disposed on the sheath body, (iii) the cable, and (iv) a conductor electrically coupled to the recording electrode and extending in the cable.

Example 69: A method comprising: providing the assembly of any one of Examples 62 to 68; disposing the lower portion of the body of the burr hole device in a burr hole of a subject such that the upper flange portion rests on a skull of the subject; inserting the cranial port device in the cavity of the burr hole device; and inserting the catheter or a catheter connector of the cranial port device through the opening of the lower portion of the body of the burr hole device.

Example 70: The method of Example 69, further comprising coupling the catheter to the catheter connector.

Example 71: The method of Example 69 or 70, further comprising: inserting the catheter through the sheath lumen; implanting a distal end of the catheter in a brain of the subject; and implanting the recording electrode of the sheath beneath the skull of the subject.

Example 72: The method of Example 71, further comprising: inserting the cable of the sheath in the bottom groove, the second top groove, or the bottom portion of the top groove; and inserting the catheter in the top groove, the first top groove, or the top portion of the top groove.

Example 73: A method comprising operatively coupling to signal apparatus the electrode disposed on the bottom surface of the lower portion of the body of the burr hole device according to any one of Examples 49 to 61 and Examples 99 to 100.

Example 74: The method of Example 69, wherein the signal apparatus is implantable signal apparatus.

Example 75: An implantable sheath comprising: a body having a proximal end and a distal end and defining a lumen from the proximal end to the distal end, wherein the lumen is configured to receive a catheter; an electrode disposed on the body; a cable extending from the body in proximity to the proximal end; and a conductor electrically coupled to the electrode and extending in the cable, wherein the cable comprises an electrical interconnect comprising a contact electrically coupled the conductor, wherein the sheath is configured to be completely implanted in a subject.

Example 76: An assembly comprising: the implantable sheath of Example 75; and the catheter, the catheter having a proximal end and a distal end, wherein the distal end of the catheter is configured to be implanted in a brain of a subject.

Example 77: An assembly comprising: a catheter configured to deliver fluid to or from a brain of a subject; a sheath comprising a lumen configured receive the catheter; and an electrode disposed on the sheath, wherein the catheter and sheath are configured such that when the catheter is received in the lumen of the sheath and a distal portion of the catheter is implanted in a brain of a subject, the electrode is positioned intracranially.

Example 78: The assembly of Example 77, wherein the sheath comprises a cable comprising a conductor electrically coupled to the electrode.

Example 79: The assembly of Example 78, further comprising a burr hole device, the burr hole device comprising: a body having a top surface and a bottom surface and defining a lumen extending from the top surface to the bottom surface, wherein the lumen is configured to receive the catheter, wherein at least a portion of the bottom surface is configured to be disposed on a surface of a skull adjacent to a burr hole in the skull of the subject, and wherein the body further defines a groove configured to receive the cable.

Example 80: A method comprising: creating a first burr hole having a first diameter in a skull of a subject wherein the skull defines an edge of the first burr hole; creating a second burr hole having a second diameter in the skull, wherein the second diameter is smaller than the first diameter, and wherein the first and second burr holes partially overlap.

Example 81: The method of Example 80, further creating a trench in the top surface of the skull, wherein the trench has a first end and a second end, wherein the first end of the trench intersects the second burr, and wherein the trench extends away from the second burr hole towards the second end.

Example 82: The method of Example 81, wherein the trench has a first depth at the first end and has a second depth at the second end, wherein the first depth is greater than the second depth.

Example 83: The method of any one of Examples 80 to 82, further comprising: inserting a catheter and sheath through the first burr hole, wherein the sheath is configured to receive the catheter and comprises an electrode and a cable comprising a conductor electrically coupled to the electrode; and inserting the cable through the second burr hole.

Example 84: The method of Example 83, as it depends from Example 81 or 82, further comprising inserting the cable in the trench.

Example 85: A burr hole device comprising a body having a top surface and a bottom surface and defining a lumen extending from the top surface to the bottom surface, wherein the lumen is configured to receive a catheter configured to allow fluid to flow to or from a brain of a subject. At least a portion of the body is configured to be inserted into a burr hole. The burr hole device further comprises an expansion member that may be deployed when the body is in the burr hole. The expansion member is configured to expand against a side of the burr hole to anchor the body within the burr hole.

Example 86: The burr hole device of Example 85, wherein the entire device is configured to be received in the burr hole.

Example 87: The burr hole device of Example 85 or 86, wherein the expansion member is deployable from a retracted state to an expanded state.

Example 88: The burr hole device of Example 87, further comprising a user actuatable member operatively couple to the expansion member, wherein actuation of the user actuatable member causes the expansion member to adapt the expanded state.

Example 89: The burr hole device of any one of Examples 85 to 88, wherein the body further defines a top groove along the top surface, wherein the top groove extends from the lumen towards a lateral edge of the top surface of the body, wherein the top groove is configured to receive the catheter, wherein the body further defines a bottom groove along the bottom surface, wherein the bottom groove extends from a lateral edge of the bottom surface towards the lumen, wherein the bottom groove is configured to receive a cable.

Example 90: The burr hole device of Example 89, wherein the body defines an upper flange portion and a lower portion, wherein the lower portion is configured to be placed within the burr hole and the upper flange portion is configured to be disposed on the surface of the skull.

Example 92: The burr hole device of Example 90, wherein the bottom groove is disposed on a bottom surface of the upper flange portion.

Example 93: The burr hole device of Example 91, wherein the lower portion of the body defines a side groove along a side of the lower portion, wherein the side groove meets with the bottom groove.

Example 94: The burr hole device of any one of Examples 90 to 93, wherein the lower portion of the body is configured to have a clearance of 1 millimeter or less when disposed in the burr hole.

Example 95: An assembly comprising: (i) the burr hole device of any one of Examples 85 to 94; the catheter; and a sheath comprising (i) a sheath body defining a sleeve lumen configured to receive the catheter, (ii) a recording electrode disposed on the sheath body, (iii) the cable, and (iv) a conductor electrically coupled to the recording electrode and extending in the cable.

Example 96: A method comprising: providing the assembly of Example 95; disposing the burr hole device in or about a burr hole of a subject; inserting the catheter through the lumen of the burr hole device; inserting the catheter through the sheath lumen; implanting a distal end of the catheter in a brain of the subject; implanting the recording electrode of the sheath beneath the skull of the subject; inserting the cable of the sheath in the bottom groove, the second top groove, or the bottom portion of the top groove; and inserting the catheter in the top groove, the first top groove, or the top portion of the top groove.

Example 97: The method of Example 96, wherein implanting the recording electrode of the sheath beneath the skull of the subject comprises implanting the electrode in the brain of the subject.

Example 98: The method of Example 96 or 97, wherein implanting a distal end of the catheter in the brain of the subject comprises implanting the distal end of the catheter in a cerebral ventricle.

Example 99: The burr hole device according to any one of Examples 49-61, further comprising one or more electrical component operatively coupled to the electrode.

Example 100: The burr hole device according to Example 99, wherein the one or more electrical component is configured to one or more of: store, transmit, and process data received from the electrode.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed is:

1. A burr hole device comprising:
a body having a top surface and a bottom surface and defining a lumen extending from the top surface to the bottom surface,
wherein the lumen is configured to receive a catheter configured to allow fluid to flow to or from a brain of a subject,
wherein at least a portion of the bottom surface is configured to be disposed on a surface of a skull adjacent to a burr hole in the skull of the subject,
wherein the body further defines a top groove along the top surface, wherein the top groove extends from the lumen towards a lateral edge of the top surface of the body, wherein the top groove is configured to receive the catheter,
wherein the body further defines a bottom groove along the bottom surface, wherein the bottom groove extends from a lateral edge of the bottom surface towards the lumen, wherein the bottom groove is configured to receive a cable.

2. The burr hole device of claim 1, wherein the body defines an upper flange portion and a lower portion, wherein the lower portion is configured to be placed within the burr hole and the upper flange portion is configured to be disposed on the surface of the skull.

3. The burr hole device of claim 2, wherein the bottom groove is disposed on a bottom surface of the upper flange portion.

4. The burr hole device of claim 3, wherein the lower portion of the body defines a side groove along a side of the lower portion, wherein the side groove meets with the bottom groove.

5. The burr hole device of claim 2, wherein the lower portion of the body is configured to have a clearance of 1 millimeter or less when disposed in the burr hole.

6. The burr hole device of claim 2, wherein the lower portion of the body has an outer diametric dimension from 10 millimeters to 20 millimeters.

7. The burr hole device of claim 2, wherein the upper flange portion of the body has an outer diametric dimension from 15 millimeters to 30 millimeters.

8. An assembly comprising:
the burr hole device of claim 1;
the catheter; and
a sheath comprising (i) a sheath body defining a sleeve lumen configured to receive the catheter, (ii) a recording electrode disposed on the sheath body, (iii) the cable, and (iv) a conductor electrically coupled to the recording electrode and extending in the cable.

9. A method comprising:
providing the assembly of claim 8;
disposing the burr hole device in or about a burr hole of a subject;
inserting the catheter through the lumen of the burr hole device;
inserting the catheter through the sheath lumen;
implanting a distal end of the catheter in a brain of the subject;
implanting the recording electrode of the sheath beneath the skull of the subject;
inserting the cable of the sheath in the bottom groove, the second top groove, or the bottom portion of the top groove; and
inserting the catheter in the top groove, the first top groove, or the top portion of the top groove.

10. The method of claim 9, wherein implanting the recording electrode of the sheath beneath the skull of the subject comprises implanting the electrode in the brain of the subject.

11. The method of claim 9, wherein implanting a distal end of the catheter in the brain of the subject comprises implanting the distal end of the catheter in a cerebral ventricle.

12. An assembly comprising:
the burr hole device of claim 2;
the catheter; and
a sheath comprising (i) a sheath body defining a sleeve lumen configured to receive the catheter, (ii) a recording electrode disposed on the sheath body, (iii) the cable, and (iv) a conductor electrically coupled to the recording electrode and extending in the cable.

13. The method of claim 12, wherein implanting the recording electrode of the sheath beneath the skull of the subject comprises implanting the electrode in the brain of the subject.

14. The method of claim 12, wherein implanting a distal end of the catheter in the brain of the subject comprises implanting the distal end of the catheter in a cerebral ventricle.
